(19) United States
(12) Patent Application Publication
(10) Pub. No.: US 2015/0122647 A1
(43) Pub. Date: May 7, 2015

(54) ENZYME MATRICES FOR USE WITH ETHYLENE OXIDE STERILIZATION

(71) Applicant: Medtronic MiniMed, Inc., Northridge, CA (US)

(72) Inventors: Rajiv Shah, Rancho Palos Verdes, CA (US); Qingling Yang, Northridge, CA (US); Robert C. Mucic, Glendale, CA (US); Jenn-Hann Larry Wang, Northridge, CA (US)

(21) Appl. No.: 14/532,364

(22) Filed: Nov. 4, 2014

Related U.S. Application Data
(63) Continuation-in-part of application No. 14/074,248, filed on Nov. 7, 2013.

(51) Int. Cl. G01N 27/327; C12Q 1/00

(52) U.S. Cl. CPC G01N 27/3271; C12Q 1/006; C12Q 1/002
USPC 204/403.14; 435/182; 422/34; 427/2.12

(57) ABSTRACT

The invention pertains to analyte sensors designed to include layered compositions that provide these sensors with enhanced functional and/or material properties including, for example, resistance to damage caused by ethylene oxide during sterilization processes. Embodiments of the invention include polyvinyl alcohol N-methyl-4(4′-formylstyryl)pyridinium (SbQ) polymer materials and methods for employing such materials during the ethylene oxide sterilization of glucose sensors.

### CROSS REFERENCE TO RELATED APPLICATIONS

This application is a continuation-in-part application which claims priority from U.S. patent application Ser. No. 14/074,248 filed Nov. 7, 2013, the contents of which are incorporated herein by reference.

### TECHNICAL FIELD

The present invention relates to methods and materials useful for analyte sensor systems, such as glucose sensors used in the management of diabetes.

### BACKGROUND OF THE INVENTION

Medical device companies, hospitals, and other health facilities employ a variety of technologies to control both infection and contamination. While compositions such as germicides, antiseptics and bacteriostats are effective in controlling widespread growth of biological contaminants, they do not go as far as to completely eliminate these agents. For this reason, sterilization processes such as those that use heat, radiation, or chemical agents such as ethylene oxide are employed to ensure total eradication of microorganisms. Ethylene oxide (EtO) sterilization is a common process that generally involves placing an item in a chamber and subjecting it to ethylene oxide vapor. Because ethylene oxide gas readily diffuses through commonly employed packaging materials and is highly effective in killing microorganisms at temperatures well below those required for heat sterilization techniques, it enables efficient sterilization of many items, particularly those which cannot withstand heat sterilization. When used properly, ethylene oxide is lethal to all known microorganisms at ordinary temperature. Rapid growth in the use of sterile, disposable medical devices is just one consequence of gaseous sterilization with agents such as ethylene oxide.

Analyte sensors are one class of medical devices typically sterilized prior to use. However, many analyte sensors include sensitive components that convert interactions with analytes into detectable signals that can be correlated with the concentrations of the analyte. For example, amperometric glucose sensors incorporate electrodes coated with layers of materials such as glucose oxidase (GOx), an enzyme that catalyzes the reaction between glucose and oxygen to yield gluconic acid and hydrogen peroxide (H₂O₂). The H₂O₂ formed in this reaction alters an electrode current to form a detectable and measurable signal. Based on the signal, the concentration of glucose in the individual can then be measured.

When devices are sterilized with ethylene oxide, problems can arise if the ethylene oxide reacts with, and inhibits the activity of one or more sensitive components of the device, such as the enzyme glucose oxidase in amperometric glucose sensors. Such problems can prevent the effective use of ethylene oxide sterilization procedures on such devices. Methods and materials designed to address such challenges in this technology are therefore desirable.

### SUMMARY OF THE INVENTION

The invention disclosed herein includes polymeric compositions for use in layered sensor designs, methods form making and using such compositions as well as sensor systems that utilize such compositions. Embodiments of the invention include polyvinyl alcohol-styrylpyridinium (PVA-SbQ) compositions having a constellation of material properties that make them particularly useful for implantable glucose sensors of the type worn by diabetic individuals. As discussed below, working embodiments of the invention include amperometric glucose sensors designed to include layered compositions that provide these sensors with enhanced functional and/or material properties including, for example, protection form damage caused by ethylene oxide during sterilization processes.

The invention disclosed herein has a number of embodiments. Typical embodiments of the invention include methods of making an analyte sensor apparatus. In such embodiments, one can dispose a working electrode, a reference electrode, and a counter electrode on a base layer, and then dispose an analyte sensing layer over the working electrode, wherein the analyte sensing layer comprises glucose oxidase disposed within a polyvinyl alcohol polymer comprising N-methyl-4(4′-formylstyryl)pyridinium (PVA-SbQ). In the working embodiments of the invention that are disclosed herein, the PVA-SbQ polymer is selected for its ability to inhibit damage to glucose oxidase by ethylene oxide. Typically these methods further include disposing an analyte modulating layer disposed over the analyte sensing layer, wherein the analyte modulating layer modulates the diffusion of analyte therethrough.

Embodiments of the invention include methods where the constituents or size of the analyte sensor layers are controlled in order to optimize one or more characteristics that are desired for a certain sensor and/or sensing environment. For example, in some embodiments of the invention, the analyte sensing layer comprises PVA-SbQ in an amount from 5% to 15% by weight. In some embodiments of the invention, the analyte sensing layer comprises glucose oxidase in an amount from 10 KU/mL to 20 KU/mL. In some embodiments of the invention, the analyte sensing layer comprises polyvinyl alcohol having a molecular weight from 25 kilodaltons to 125 kilodaltons. In some embodiments of the invention, the analyte sensing layer comprises 1.0% to 4.0% N-methyl-4(4′-formylstyryl)pyridinium. In addition, in some embodiments of the invention, the analyte sensing layer is between 4 and 12 microns in thickness. Optionally, the analyte sensor apparatus comprises a further layer disposed over the analyte sensing layer. In some embodiments of the invention, the further layer comprises PVA-SbQ. In some embodiments of the invention, the further layer comprises a hydrophilic polyurethane. In some embodiments of the invention, the further layer does not include an albumin. In addition, in some embodiments of the invention, the further layer is between 1 and 3 microns in thickness.

Other embodiments of the invention include methods of inhibiting damage to glucose oxidase caused by ethylene oxide vapor during a sterilization process, the method comprising disposing the glucose oxidase within a matrix comprising a polyvinyl alcohol polymer comprising N-methyl-4(4′-formylstyryl)pyridinium (PVA-SbQ), so that damage to glucose oxidase is inhibited. Typically in these methods, the glucose oxidase is disposed within an analyte sensor apparatus, wherein the analyte sensor apparatus comprises: a base layer; a working electrode, a reference electrode, and a counter electrode disposed on the base layer an analyte sensing layer disposed over the working electrode, wherein the analyte sensing layer comprises the glucose oxidase disposed within the PVA-SbQ; and an analyte modulating layer disposed over the analyte sensing layer, wherein the analyte modulating layer modulates the diffusion of analyte therethrough.

Another embodiment of the invention is a method of inhibiting microbial growth on an analyte sensor apparatus. Typically such methods comprise exposing the analyte sensor apparatus to an ethylene oxide vapor so as to contact a microorganism present on the analyte sensor apparatus or a container in which the analyte sensor apparatus is disposed; and then allowing the ethylene oxide to alkylate DNA of the microorganism, thereby inhibiting microbial growth. In these methods, the analyte sensor apparatus comprises a base layer, a working electrode, a reference electrode, and a counter electrode disposed on the base layer, an analyte sensing layer disposed over the working electrode, wherein the analyte sensing layer comprises glucose oxidase disposed within a polyvinyl alcohol polymer comprising N-methyl-4(4′-formylstyryl)pyridinium (PVA-SbQ); and an analyte modulating layer disposed over the analyte sensing layer, wherein the analyte modulating layer modulates the diffusion of analyte therethrough.

Yet another embodiment of the invention is a composition of matter comprising an analyte sensor apparatus and ethylene oxide vapor. In this composition, the sensor apparatus includes a base layer, a working electrode, a reference electrode, and a counter electrode disposed on the base layer, an analyte sensing layer disposed over the working electrode, wherein the analyte sensing layer comprises glucose oxidase entrapped within either a polyvinyl alcohol polymer comprising N-methyl-4(4′-formylstyryl)pyridinium (SbQ); or a hydrophilic polyurethane. Typically in these embodiments, the sensor includes an analyte modulating layer disposed over the analyte sensing layer, wherein the analyte modulating layer modulates the diffusion of analyte therethrough.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 shows a sensor design comprising an amperometric analyte sensor formed from a plurality of planar layered elements.

FIG. 2 provides a perspective view illustrating a subcutaneous sensor insertion set, a telemetered characteristic monitor transmitter device, and a data receiving device embodying features of the invention.

FIGS. 3A-3C provide graphical data showing how the polymer matrices disclosed herein can significantly improved the resistance of polypeptides such as glucose oxidase to EtO degradation during sterilization processes, thereby preserving glucose oxidase activity as other performance characteristics such as O₂ dependency as well as sensor uniformity. FIG. 3A shows the glucose response profile of a sensor the has not been EtO sterilized; FIG. 3B shows the poor glucose response profile following EtO sterilization of a sensor that does not incorporate the PVA-SbQ materials disclosed herein; and FIG. 3C shows the improved glucose response profile following EtO sterilization of a sensor that incorporates the PVA-SbQ materials disclosed herein. FIG. 3B shows that GOX immobilized via typical methods does not survive EtO sterilization, with signal intensity dropping, variability increasing, and behavior during oxygen response testing indicating a loss of performance. FIG. 3C shows that sterilization of GOX using compositions disclosed herein, and/or applying polymeric protective layers offer stability to the sensors and preserve their desired oxygen response.

FIGS. 4A-4B show data from studies of sensors in an in vitro testing system (SITS) that is designed to mimic in vivo conditions. In this system, sensor current is measured periodically in the presence of known concentrations of glucose and glucose values are then correlated with Isig, that is sensor current (in μA). These graphs provide data (Isig over periods of time) from experiments using sensors constructed to include different GLM analyte modulating compositions. FIGS. 4A-4B provide graphical data showing test results of ETO sterilized glucose sensors having analyte modulating layers formed without 4,4′-methylene bis(phenyl isocyanate) (FIG. 4A) as compared to sensors having analyte modulating layers formed with 10% 4,4′-methylene bis(phenyl isocyanate (FIG. 4B) The data presented in FIGS. 4A & 4B show that the sensor to sensor signal variation is much less in sensors having analyte modulating layers formed without with 10% 4,4′-methylene bis(phenyl) isocyanate. This data shows how the inclusion of diisocyanate compounds comprising a phenyl moiety in the analyte modulating layers can significantly improve the resistance of polypeptides such as glucose oxidase to EtO degradation during sterilization processes.

### DETAILED DESCRIPTION OF THE INVENTION

Unless otherwise defined, all terms of art, notations, and other scientific terms or terminology used herein are intended to have the meanings commonly understood by those of skill in the art to which this invention pertains. In some cases, terms with commonly understood meanings may be defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. A number of terms are defined below.

All numbers recited in the specification and associated claims that refer to values that can be numerically characterized with a value other than a whole number (e.g. a unit of measurement such as a concentration of a component in a composition) are understood to be modified by the term “about”. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Furthermore, all publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

The term “analyte” as used herein is a broad term and is used in its ordinary sense, including, without limitation, to refer to a substance or chemical constituent in a fluid such as a biological fluid (for example, blood, interstitial fluid, cerebral spinal fluid, lymph fluid or urine) that can be analyzed. Analytes can include naturally occurring substances, artificial substances, metabolites, and/or reaction products. In common embodiments, the analyte is glucose. However, embodiments of the invention can be used with sensors designed for detecting a wide variety other analytes. Illustrative analytes include but are not limited to, lactate as well as salts, sugars, proteins fats, vitamins and hormones that naturally occur in vivo (e.g. in blood or interstitial fluids). The analyte can be naturally present in the biological fluid or endogenous; for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin. The metabolic products of drugs and pharmaceutical compositions are also contemplated analytes.

The term “sensor” for example in “analyte sensor,” is used in its ordinary sense, including, without limitation, means used to detect a compound such as an analyte. A “sensor system” includes, for example, elements, structures and architectures (e.g. specific 3-dimensional constellations of molecular elements) designed to facilitate sensor use and function. Sensor systems can include, for example, compositions such as those having selected material properties, as well as electronic components such as elements and devices used in signal detection and analysis (e.g. current detectors, monitors, processors and the like).

As discussed in detail below, embodiments of the invention relate to the use of an electrochemical sensor that measures a concentration of an analyte of interest or a substance indicative of the concentration or presence of the analyte in fluid. In some embodiments, the sensor is a continuous device, for example a subcutaneous, transdermal, or intravascular device. In some embodiments, the device can analyze a plurality of intermittent blood samples. The sensor embodiments disclosed herein can use any known method, including invasive, minimally invasive, and non-invasive sensing techniques, to provide an output signal indicative of the concentration of the analyte of interest. Typically, the sensor is of the type that senses a product or reactant of an enzymatic reaction between an analyte and an enzyme in the presence of oxygen as a measure of the analyte in vivo or in vitro. Such sensors typically comprise a membrane surrounding the enzyme through which an analyte migrates. The product is then measured using electrochemical methods and thus the output of an electrode system functions as a measure of the analyte.

Embodiments of the invention disclosed herein provide sensors of the type used, for example, in subcutaneous or transcutaneous monitoring of blood glucose levels in a diabetic patient. A variety of implantable, electrochemical biosensors have been developed for the treatment of diabetes and other life-threatening diseases. Many existing sensor designs use some form of immobilized enzyme to achieve their bio-specificity. Embodiments of the invention described herein can be adapted and implemented with a wide variety of known electrochemical sensors, including for example, U.S. Patent Application No. 20050115832, U.S. Pat. Nos. 6,001,067, 6,702,857, 6,212,416, 6,119,028, 6,400,974, 6,595,919, 6,141,573, 6,122,536, 6,512,939 5,605,152, 4,431,004, 4,703,756, 6,514,718, 5,985,129, 5,390,691, 5,391,250, 5,482,473, 5,299,571, 5,568,806, 5,494,562, 6,120,676, 6,542,765, 7,033,336 as well as PCT International Publication Numbers WO 01/58348, WO 04/021877, WO 03/034902, WO 03/035117, WO 03/035891, WO 03/023388, WO 03/022128, WO 03/022352, WO 03/023708, WO 03/036255, WO03/036310 WO 08/042,625, and WO 03/074107, and European Patent Application EP 1153571, the contents of each of which are incorporated herein by reference.

### Illustrative Embodiments of the Invention and Associated Characteristics

EtO sterilization requires evacuation of a sterilization chamber, the introduction of moisture, the introduction of the EtO gas (e.g. either in the pure state or as a mixture with an inert gas), and keeping the internal pressure of the chamber lower than one atmosphere to prevent leakage of the EtO to the external environment. After some specified exposure time, EtO is purged from the chamber and the chamber is flooded with filtered sterile air to remove any residual EtO.

The EtO high chemical reactivity, as expressed by the high energy of its exergonic combustion reaction, in combination with its high diffusivity, is of major importance for the inactivation of microorganisms. EtO is a direct alkylating agent that does not require metabolic activation, and its microbiologic inactivation properties are considered to be the result of its powerful alkylation reaction with cellular constituents of organisms, such as nucleic acid and functional proteins, including enzymes, which leads to consequent denaturation. The addition of alkyl groups to proteins (e.g. glucose oxidase), DNA, and RNA in microorganisms by binding to the sulfhydryl and hydroxyl, amino, and carboxyl groups, prevents normal cellular metabolism and ability to reproduce, which render affected microbes nonviable. These chemical moieties are not present in most of the plastic medical device composition; therefore, exposure to EtO does not cause them similar structural changes. The majority of plastics are unaffected by EtO sterilization treatment, but some can absorb EtO and these must be treated to eliminate any EtO before use.

Embodiments of the invention disclosed herein provide analyte sensors designed to include layered compositions that provide these sensors with enhanced functional and/or material properties including, for example, resistance to damage to polypeptides such as glucose oxidase caused by ethylene oxide during sterilization processes. As discussed in detail below, typical embodiments of the invention relate to methods and materials designed for use in the sterilization a sensor that measures a concentration of an aqueous analyte of interest or a substance indicative of the concentration or presence of the analyte in vivo (e.g. glucose).

The invention disclosed herein has a number of embodiments. Typical embodiments of the invention include methods of making an analyte sensor apparatus. In such embodiments, one can dispose a working electrode, a reference electrode, and a counter electrode on a base layer, and then dispose an analyte sensing layer over the working electrode, wherein the analyte sensing layer comprises glucose oxidase disposed within a polyvinyl alcohol polymer comprising N-methyl-4(4′-formylstyryl)pyridinium (PVA-SbQ). In some embodiments of the invention, sensing layer comprises glucose oxidase disposed within a hydrophilic polyurethane (e.g. either alone or in combination with PVA-SbQ). In the working embodiments of the invention that are disclosed herein, the PVA-SbQ polymer (and/or hydrophilic polyurethane) is selected for its ability to inhibit damage to glucose oxidase by ethylene oxide.

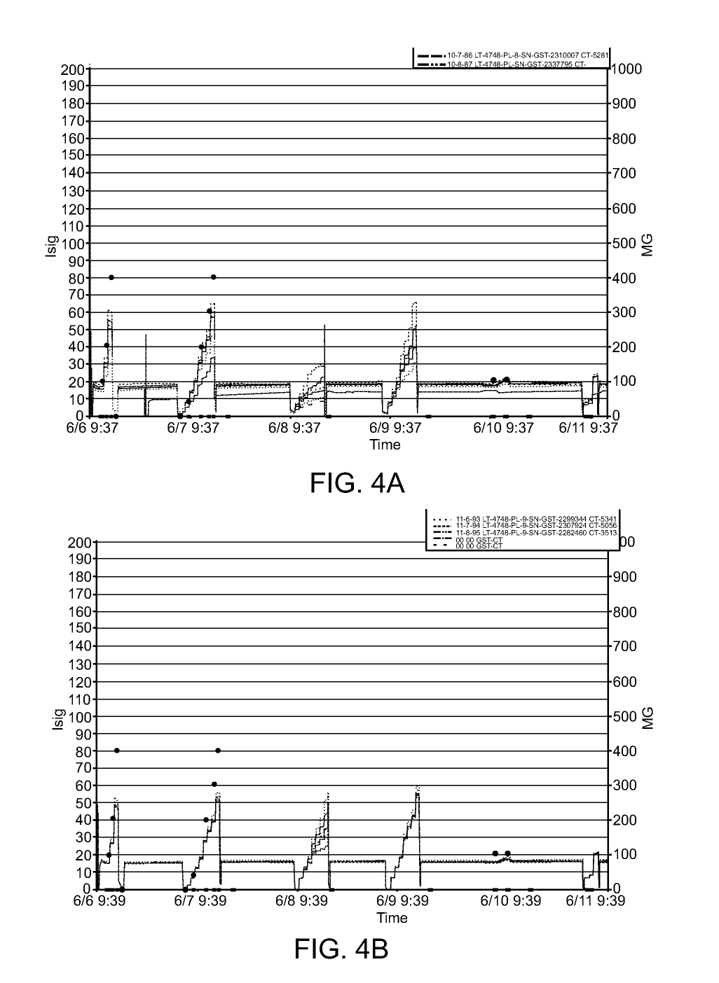

Typical methods can further include disposing an analyte modulating layer disposed over the analyte sensing layer, wherein the analyte modulating layer modulates the diffusion of analyte therethrough. In certain embodiments of the invention, the analyte modulating layer is formed from a composition that includes a diisocyanate comprising a phenyl moiety. As shown in FIG. 4, the use of diisocyanate compounds comprising a phenyl moiety in the analyte modulating layer can further protect sensors from damage by ethylene oxide sterilization. Without being bound by a particular theory or mechanism of action, it is believed that phenyl moieties may scavenge free radicals that are produced by the sterilization process, compounds that can damage the sensor components such as glucose oxidase. In the embodiments shown in FIG. 4B, the analyte modulating layer is formed from a mixture comprising 10%-40% of a diisocyanate compound comprising a phenyl moiety, e.g. diisocyanate is 4,4′-methylene bis(phenyl isocyanate), CASRN 101-68-8.

Optionally in these sensors, the analyte modulating layer comprises a polyurethane/polyurea polymer formed from a mixture comprising a diisocyanate, a hydrophilic polymer comprising a hydrophilic diol or hydrophilic diamine; and a siloxane having an amino, hydroxyl or carboxylic acid functional group at a terminus. In embodiments of the invention, analyte modulating layer can comprise a branched acrylate polymer formed from a mixture comprising a butyl, propyl, ethyl or methyl-acrylate, an amino-acrylate, a siloxane-acrylate; and a poly(ethylene oxide)-acrylate.

Embodiments of the invention include methods where the constituents or size of the analyte sensor layer are controlled in order to optimize one or more desirable characteristics. For example, in some embodiments of the invention, the analyte sensing layer comprises PVA-SbQ in an amount from 5% to 15% by weight. In some embodiments of the invention, the analyte sensing layer comprises glucose oxidase in an amount from 10 KU/mL to 20 KU/mL. In some embodiments of the invention, the analyte sensing layer comprises polyvinyl alcohol having a molecular weight from 25 kilodaltons to 125 kilodaltons. In some embodiments of the invention, the analyte sensing layer comprises 1.0% to 4.0% N-methyl-4(4′-formylstyryl)pyridinium. In addition, in some embodiments of the invention, the analyte sensing layer is between 4 and 12 microns in thickness. Optionally, the analyte sensor apparatus comprises a further layer disposed over the analyte sensing layer. In some embodiments of the invention, the further layer comprises PVA-SbQ. In some embodiments of the invention, the further layer comprises a hydrophilic polyurethane. In some embodiments of the invention, the further layer does not include an albumin. In addition, in some embodiments of the invention, the further layer is between 1 and 3 microns in thickness.

Other embodiments of the invention include methods of inhibiting damage to glucose oxidase caused by ethylene oxide vapor during a sterilization process, the method comprising disposing the glucose oxidase within a matrix comprising a polyvinyl alcohol polymer comprising N-methyl-4(4′-formylstyryl)pyridinium (PVA-SbQ), so that damage to glucose oxidase is inhibited. Typically in these methods, the glucose oxidase is disposed within an analyte sensor apparatus, wherein the analyte sensor apparatus comprises: a base layer; a working electrode, a reference electrode, and a counter electrode disposed on the base layer an analyte sensing layer disposed over the working electrode, wherein the analyte sensing layer comprises the glucose oxidase disposed within the PVA-SbQ; and an analyte modulating layer disposed over the analyte sensing layer, wherein the analyte modulating layer modulates the diffusion of analyte therethrough.

In some methods of inhibiting damage to glucose oxidase, the analyte sensing layer comprises PVA-SbQ in an amount from 5% to 15% by weight and/or comprises glucose oxidase in an amount from 10 KU/mL to 20 KU/mL, and/or comprises polyvinyl alcohol having a molecular weight from 25 kilodaltons to 125 kilodaltons, and/or comprises 1.0% to 4.0% N-methyl-4(4′-formylstyryl)pyridinium; and/or is between 4 and 12 microns in thickness. Optionally in these methods, the analyte sensor apparatus comprises a further layer disposed over the analyte sensing layer, wherein the further layer comprises PVA-SbQ and/or comprises a hydrophilic polyurethane and/or does not include an albumin; and/or is between 1 and 3 microns in thickness.

In some embodiments of the invention, layers of the sensor are modified, for example by glutaraldehyde crosslinking or a plasma deposition process. In an illustrative embodiment, a plasma deposition process is used to promote adhesion between one or more layers (e.g. after a hydrophilic polyurethane layer in order to make a dry process without glutaraldehyde). For example, one can form an adhesion promoting layer over the hydrophilic polyurethane and/or analyte sensing layer etc., wherein the adhesion promoting layer comprises hexamethyldisiloxane and is formed over a preceding layer using a plasma vapor deposition process (e.g. in the absence of a chemical crosslinking agent such as glutaraldehyde). Such plasma vapor deposition process are described, for example, in U.S. patent application Ser. No. 13/541,262, the contents of which are incorporated by reference.

Yet another embodiment of the invention is a method of inhibiting microbial growth on an analyte sensor apparatus. Typically such methods comprise exposing the analyte sensor apparatus to an ethylene oxide vapor so as to contact a microorganism present on the analyte sensor apparatus or a container in which the analyte sensor apparatus is disposed; and then allowing the ethylene oxide to alkylate DNA of the microorganism, thereby inhibiting microbial growth. In these methods, the analyte sensor apparatus comprises a base layer, a working electrode, a reference electrode, and a counter electrode disposed on the base layer, an analyte sensing layer disposed over the working electrode, wherein the analyte sensing layer comprises glucose oxidase disposed within a polyvinyl alcohol polymer comprising N-methyl-4(4′-formylstyryl)pyridinium (PVA-SbQ); and an analyte modulating layer disposed over the analyte sensing layer, wherein the analyte modulating layer modulates the diffusion of analyte therethrough.

In some methods for inhibiting microbial growth on an analyte sensor apparatus, the analyte sensing layer comprises PVA-SbQ in an amount from 5% to 15% by weight and/or comprises glucose oxidase in an amount from 10 KU/mL to 20 KU/mL, and/or comprises polyvinyl alcohol having a molecular weight from 25 kilodaltons to 125 kilodaltons, and/or comprises 1.0% to 4.0% N-methyl-4(4′-formylstyryl)pyridinium; and/or is between 4 and 12 microns in thickness. Optionally in these methods, the analyte sensor apparatus comprises a further layer disposed over the analyte sensing layer, wherein the further layer comprises PVA-SbQ and/or comprises a hydrophilic polyurethane and/or does not include an albumin; and/or is between 1 and 3 microns in thickness.

In some methods for inhibiting microbial growth on an analyte sensor apparatus, the analyte sensing layer comprises PVA-SbQ in an amount from 5% to 15% by weight and/or comprises glucose oxidase in an amount from 10 KU/mL to 20 KU/mL, and/or comprises polyvinyl alcohol having a molecular weight from 25 kilodaltons to 125 kilodaltons, and/or comprises 1.0% to 4.0% N-methyl-4(4′-formylstyryl)pyridinium; and/or is between 4 and 12 microns in thickness. Optionally in these methods, the analyte sensor apparatus comprises a further layer disposed over the analyte sensing layer, wherein the further layer comprises PVA-SbQ and/or comprises a hydrophilic polyurethane and/or does not include an albumin; and/or is between 1 and 3 microns in thickness. Optionally in these methods, the analyte sensing layer comprises PVA-SbQ in an amount from 5% to 15% by weight and/or comprises glucose oxidase in an amount from 10 KU/mL to 20 KU/mL, and/or comprises polyvinyl alcohol having a molecular weight from 25 kilodaltons to 125 kilodaltons, and/or comprises 1.0% to 4.0% N-methyl-4(4′-formylstyryl)pyridinium; and/or is between 4 and 12 microns in thickness. Optionally in these methods, the analyte sensor apparatus comprises a further layer disposed over the analyte sensing layer, wherein the further layer comprises PVA-SbQ and/or comprises a hydrophilic polyurethane and/or does not include an albumin; and/or is between 1 and 3 microns in thickness.

Typically in these methods for inhibiting microbial growth on an analyte sensor apparatus, the sensor apparatus comprises a further layer disposed over the analyte sensing layer, wherein the further layer comprises PVA-SbQ and/or comprises a hydrophilic polyurethane and/or does not include an albumin; and/or is between 1 and 3 microns in thickness. Optionally this method uses ethylene oxide vapor in a concentration range from 50 to 1,500 mg/L. Optionally this method uses humidity in a range from 30% to 90%. Optionally this method is performed at a temperature from 25-55° C. Optionally, the method is performed for at least 2 hours. Optionally in these methods, the analyte modulating layer comprises a polyurethane/polyurea polymer formed from a mixture comprising a diisocyanate, a hydrophilic polymer comprising a hydrophilic diol or hydrophilic diamine; and a siloxane having an amino, hydroxyl or carboxylic acid functional group at a terminus. In embodiments of the invention, analyte modulating layer can comprises a branched acrylate polymer formed from a mixture comprising a butyl, propyl, ethyl or methyl-acrylate, an amino-acrylate, a siloxane-acrylate; and a poly(ethylene oxide)-acrylate.

Yet another embodiment of the invention is a composition of matter comprising an analyte sensor apparatus and ethylene oxide vapor. In this composition, the sensor apparatus includes a base layer, a working electrode, a reference electrode, and a counter electrode disposed on the base layer, an analyte sensing layer disposed over the working electrode, wherein the analyte sensing layer comprises glucose oxidase entrapped within either a polyvinyl alcohol polymer comprising N-methyl-4(4′-formylstyryl)pyridinium (SbQ); or a hydrophilic polyurethane. Typically in these embodiments, the sensor includes an analyte modulating layer disposed over the analyte sensing layer, wherein the analyte modulating layer modulates the diffusion of analyte therethrough.

Optionally in these compositions, the analyte sensing layer comprises PVA-SbQ in an amount from 5% to 15% by weight and/or comprises glucose oxidase in an amount from 10 KU/mL to 20 KU/mL, and/or comprises polyvinyl alcohol having a molecular weight from 25 kilodaltons to 125 kilodaltons, and/or comprises 1.0% to 4.0% N-methyl-4(4′-formylstyryl)pyridinium; and/or is between 4 and 12 microns in thickness. Optionally in these compositions, the analyte sensor apparatus comprises a further layer disposed over the analyte sensing layer, wherein the further layer comprises PVA-SbQ and/or comprises a hydrophilic polyurethane and/or does not include an albumin; and/or is between 1 and 3 microns in thickness; and/or is between 4 and 12 microns in thickness. In certain embodiments of the invention, the composition does not comprise a separate protein layer comprising albumin and/or the analyte sensing layer does not comprise an albumin. In some compositions, the analyte sensing layer comprises glucose oxidase trapped within a hydrophilic polyurethane.

As noted above, compositions used in layered analyte sensors typically include functionally active polypeptides such as glucose oxidase (e.g. compositions comprising glucose oxidase combined with a carrier protein such as albumin). Such compositions exhibit a tendency to develop cracks when using certain formulations of constituents. Layers in amperometric glucose sensors become prone to developing cracks (e.g. as the concentration of glucose oxidase in the analyte sensing layer is increased), features that that can compromise sensor function. For example, cracks can cause the layered compositions to peel away from other sensor elements (e.g. electrodes, other proximal layered compositions and the like). Such layer delamination, a phenomenon that is difficult to control, can lead to unreliable and/or inconsistent sensor performance.

The invention disclosed herein uses selected polymeric enzyme matrices that designed to address problems associated with layer delamination in analyte sensors. One illustrative embodiment is an amperometric analyte sensor apparatus comprising a base layer that includes a working electrode, a reference electrode, and a counter electrode. In this embodiment an analyte sensing layer is disposed over the working electrode and this analyte sensing layer comprises glucose oxidase entrapped with a polymer comprising a polyvinyl alcohol polymer that is coupled to N-methyl-4(4′-formylstyryl)pyridinium (see, e.g. CAS No. 74401-04-0). As discussed below, such embodiments of the invention having such layers exhibit reduced sensor layer cracking and/or delamination. In this context, the term “entrapped” means the occlusion of glucose oxidase within a PVA polymeric network in a manner that allows fluids to pass through but retains the glucose oxidase. This entrapment method/system differs from methods/systems that covalently couple glucose oxidase to a polymer. In this embodiment, an analyte modulating layer is disposed over this analyte sensing layer and functions to modulates the diffusion analytes such as glucose through the layers of the analyte sensor. Optionally, the sensor apparatus also comprises an interference rejection layer disposed over the electrode; an adhesion promoting layer disposed between the analyte sensing layer and the analyte modulating layer; a protein layer disposed on the analyte sensing layer; and/or a cover layer disposed over the analyte modulating layer. In certain embodiments of the invention, the analyte sensor comprises a further layer disposed over the analyte modulating layer, wherein the further layer comprises polyvinyl alcohol polymer.

Embodiments of the invention can utilize different configurations of elements and/or different composition constituents and/or composition forming processes in their analyte sensing layers in order to enhance their material properties. For example, certain embodiments of the invention involve further crosslinking procedures (e.g. to further localize entrapped glucose oxidase enzymes to a particular region of a PVA matrix). Briefly, as is known in the art, crosslinking is the process of chemically joining two or more molecules by a covalent bond. Crosslinking reagents (or crosslinkers) are molecules that contain two or more reactive ends/moieties capable of chemically attaching to specific functional groups (e.g. primary amines, sulfhydryls, etc.) on proteins or other molecules. Attachment between two groups on a single protein results in intramolecular bonds that stabilize the protein tertiary or quaternary structure. Attachment between groups on two different proteins results in intermolecular bonds/bridges that can stabilize a protein-protein interaction. In compositions formed from a mixture of purified proteins (e.g. human serum albumin and an enzyme such as glucose oxidase), the intermolecular bonds/bridges create a specific conjugate that can be useful in detection procedures.

Glutaraldehyde is a common crosslinking agent. The reaction of glutaraldehyde with enzymes to give insoluble products has been extensively studied, and the reaction is known to be pH-dependent. In this context, the optimum pH for glutaraldehyde insolubilization can vary from protein to protein. Protein isoelectric points (pIs) can be the pH values for the most rapid insolubilization for some but not all proteins. The existence of an optimal crosslinking pH suggests an important role for protein charge on the intermolecular crosslinking required for insolubilization. Such charges on proteins may regulate crosslinking, which, for example, may be maximal when the repulsive charges are minimized. Similarly, the ionic strength of the composition/medium can also play some role (e.g. the lower, the better for some systems).

Certain embodiments of the invention include entrapped and crosslinked polypeptides such as glucose oxidase crosslinked to polyvinyl alcohol (PVA, see, e.g. CAS number 9002-89-5) polymers. As is known in the art, polyvinyl alcohol reacts with aldehydes to form water insoluble polyacetals. In a pure PVA medium having a pH around 5.0, polymer reaction with dialdehydes is expected to form an acetal cross-linked structure. In certain embodiments of the invention, such crosslinking reactions are performed using a chemical vapor deposition (CVD) process. Due to the acidity of the PVA polymer solution, crosslinking reactions in CVD systems are simple and routine. Moreover, acidic conditions can be created by introducing compounds such as acetic acid into glutaraldehyde solutions, so a CVD system can provide an acid vapor condition. In addition the pH of the polymer medium can be adjusted by adding acidic compounds such as citric acid, polymer additives such as polylysine, HBr and the like.

Embodiments of the invention include compositions having properties that make them particularly well suited for use in ambulatory glucose sensors of the type worn by diabetic individuals. Such embodiments of the invention include PVA-SbQ compositions for use in layered analyte sensor structures that comprise between 1 mol % and 12.5 mol % SbQ. In certain embodiments of the invention that are adapted or use in glucose sensors, the constituents in this layer are selected so that the molecular weight of the polyvinyl alcohol is between 30 kilodaltons and 150 kilodaltons and the SbQ in the polyvinyl alcohol is present in an amount between 1 mol % and 4 mol %. In some embodiments of the invention the analyte sensing layer is formed to comprise from 5% to 12% PVA by weight. In some embodiments of the invention the analyte sensing layer is formed to comprise glucose oxidase in an amount from 10 KU/mL to 20 KU/mL. In some embodiments of the invention the analyte sensing layer is formed to comprise human serum albumin in an amount from 1% to 5% by weight.

As noted above, in some embodiments of the invention, the polyvinyl alcohol polymer can be covalently coupled to entrapped glucose oxidase via a glutaraldehyde crosslinking moiety (see, e.g. U.S. Pat. No. 7,678,767, the contents of which are incorporated by reference). In some embodiments of the invention, glucose oxidase is covalently coupled to another polypeptide within the layer, for example via a glutaraldehyde crosslinking moiety. Illustrative second polypeptides include glucose oxidase (i.e. two glucose oxidase polypeptides crosslinked together) and/or albumin (i.e. a glucose oxidase polypeptide crosslinked to a human serum albumin polypeptide). Optionally, the crosslinked analyte sensing layer is of a specific dimension or shape, for example less than 5, 4, 3, 2 or 1 microns in thickness. In another example, the analyte sensing layer is formed to be between 1 μm and 8 μm in thickness (e.g. between 2 μm and 5 μm).

Embodiments of the invention include analyte sensing layers selected for their ability to provide desirable characteristics for implantable sensors. In certain embodiments of the invention an amount or ratio of PVA within the composition is used to modulate the water adsorption of the composition, the crosslinking density of the composition etc. Such formulations can readily be evaluated for their effects on phenomena such as H₂O adsorption, sensor isig drift and in vivo start up profiles. Sufficient H₂O adsorption can help to maintain a normal chemical and electrochemical reaction within amperometric analyte sensors. Consequently, it is desirable to form such sensors from compositions having an appropriate hydrophilic chemistry. In this context, the PVA-GOx compositions disclosed herein can be used to create electrolyte hydrogels that are useful in internal coating/membrane layers and can also be coated on top of an analyte modulating layer (e.g. a glucose limiting membrane or “GLM”) in order to improve the biocompatibility and hydrophilicity of the GLM layer.

Embodiments of the invention also include methods for making and using the layered sensors disclosed herein. For example another embodiment of the invention is a method of making an analyte sensor apparatus. Typically such methods comprise the steps of providing a base layer on which is formed a conductive layer that includes a working electrode, a reference electrode and a counter electrode. In these methods, an analyte sensing layer is formed over the conductive layer, one that comprises glucose oxidase entrapped within a polyvinyl alcohol layer. The methods further include forming an analyte modulating layer disposed over the analyte sensing layer, wherein the analyte modulating layer includes a composition that modulates the diffusion of an analyte (e.g. glucose) therethrough. In certain embodiments of the invention, the polyvinyl alcohol polymer is further covalently coupled to glucose oxidase, for example using a process that includes a glutaraldehyde crosslinking step. Optionally, the glutaraldehyde crosslinking step is performed using a chemical vapor deposition (CVD) process, for example one that utilizes a vapor having a pH less than 6, 5 or 4.

Embodiments of these methods can include forming a plurality of additional layers over the analyte modulating layer, for example one that also includes a polyvinyl alcohol polymer that increases the biocompatibility and/or the hydrophilicity of the analyte sensor apparatus (e.g. in in vivo environments). Typically in these methods, the material comprising glucose oxidase entrapped within a polyvinyl alcohol polymer is rinsed prior to coating the composition with another layer of material (e.g. an analyte modulating layer). For example, in some embodiments of the invention, the analyte sensing layer is rinsed immediately after the glucose oxidase is entrapped within the PVA-SbQ polymer following this layer's exposure to UV light. Such embodiments of the invention can also include the steps of forming a protein layer on the analyte sensing layer and/or forming an adhesion promoting layer on the analyte sensing layer (or the protein layer); and/or forming a cover layer disposed on at least a portion of the analyte modulating layer, wherein the cover layer further includes an aperture over at least a portion of the analyte modulating layer. Optionally in these methods, the analyte modulating layer comprises a polyurethane/polyurea polymer formed from a mixture comprising a diisocyanate, a hydrophilic polymer comprising a hydrophilic diol or hydrophilic diamine; and a siloxane having an amino, hydroxyl or carboxylic acid functional group at a terminus; and/or a branched acrylate polymer formed from a mixture comprising a butyl, propyl, ethyl or methyl-acrylate, an amino-acrylate, a siloxane-acrylate; and/or a poly(ethylene oxide)-acrylate.

Yet another embodiment of the invention is a method of inhibiting delamination of a layered material within an analyte sensor apparatus that is designed to include a constellation of elements including a base layer, a working electrode, a reference electrode, and a counter electrode disposed on the base layer, an analyte sensing layer disposed over the working electrode (e.g. one less than 5, 4, 3, 2 or 1 microns in thickness); and an analyte modulating layer disposed over the analyte sensing layer. In such embodiments, the methods comprise forming the analyte sensing layer from a composition comprising glucose oxidase entrapped within a PVA-SbQ polymer. Typically in these methods, the PVA-SbQ polymer comprises between 1 mol % and 4.5 mol % SbQ, has molecular weight between 25 kilodaltons and 125 kilodaltons, and inhibits the formation of cracks in the analyte sensing layer and/or delamination of layered material within the analyte sensing apparatus. By using a layer having these selected material properties delamination of layered materials within the analyte sensor apparatus is inhibited. This methodology can work with a number of layered sensor apparatuses such as those that comprise layers such as an interference rejection layer (e.g. one disposed directly on top of the electrode surface), an adhesion promoting layer disposed between the analyte sensing layer and the analyte modulating layer; and/or a protein layer disposed on the analyte sensing layer.

Yet another embodiment of the invention is a method of sensing an analyte within the body of a mammal. Typically this method comprises implanting an analyte sensor as disclosed herein within the mammal (e.g. in the interstitial space of a diabetic individual), sensing an alteration in current at the working electrode in the presence of the analyte; and then correlating the alteration in current with the presence of the analyte, so that the analyte is sensed. While typical embodiments of the invention pertain to glucose sensors, the layered compositions disclosed herein can be adapted for use with a wide variety of devices known in the art.

Sensors formed from a variety of analyte sensing compositions under various process conditions all showed a desired Isig level with good consistency. The pO₂ effect in these sensors is very low. HMW PVAs were observed to usually produce layers/membranes with a dense structure and lower Isig than LMW PVAs. Moreover, with a 5% PVA matrix, GOx thickness did not significantly effect in vitro sensor performance. Sensors formed with analyte sensing compositions having a higher PVA content did show higher Isig and pO₂ effect (perhaps due to higher H₂O absorption and less crosslinking density). This data provides evidence that highly desirable sensor embodiments include those formed from analyte sensing compositions having about 5% PVA (typically LMW PVA) and designed to be between 1 and 7 μm in thickness. PVA molecular weights can also be selected to control the desired Isig level for in vivo uses. Additionally, the material stability provided by the analyte sensing compositions disclosed herein further benefits subsequent layer coatings disposed over this layer by providing a smoother and more uniform surface on which to dispose these coatings, thereby facilitating further layer coating adherence and lamination.

Embodiments of the invention comprise PVA-SbQ polymers having selected molecular weights and constituent ratios. In general, the structure of the PVA-SbQ materials useful in analyte sensors is one that is dense and stable enough to maintain its integrity over the time in aqueous medium (e.g. in vivo). For glucose sensor performance, a series of phenomena such as glucose permeability, temperature effect, O₂ permeability etc. are also key considerations. As part of an effort to determine PVA-SbQ material parameters that effect glucose sensor function, we performed experiments where properties of the PVA-SbQ materials (e.g. MW and SbQ content) were varied and then tested in sensors. In working embodiments of the invention, PVAs of different molecular weights were dissolved in 90-98° C. hot H₂O for 4 hours. Low molecular weight “LMW” PVA having a molecular weight range of 31 k to 50 kd can be purchased commercially (e.g. Aldrich#36313-8) as well as high molecular weight “HMW” PVA having a molecular weight range from 85 kd to 146 kd (of (Aldrich#36314-6). GOx purchased from BIOZYME at 0.1 mu/mL was mixed with PVA solution to make the desired enzyme concentration. This solution was then applied to the sensor via spin coating with 200-300 rpm of GOx solution to form 1.0 to 3 um layer, followed by application of a human serum albumin layer and a CVD process. The final analyte modulating (GLM) layer was then slot coated onto the sensor at a desired thickness.

PVA-SbQ methods and materials are known in the art (see, e.g. U.S. Pat. Nos. 7,638,157, 7,415,299 and 6,379,883, and Ichimura et al., Journal of Polymer Science Part A: Polymer Chemistry, Volume 50, Issue 19, pages 4094-4102 (2012)). For example, as is known in the art, PVA can be acetalized with N-methyl-4-(p-formyl styryl) Pyridinium methosulfate (SbQ). Photosensitive compound, 1-methyl-4-[2-(4-diethylacetylphenyl)ethenyl]pridininm methosulfate (SbQ-A salt), was synthesized from dimethyl sulfate, terephthalaldehyde mono-(diethylacetal) and 4-picoline. SbQ-A salts were reacted with poly (vinyl alcohol)s, (PVA) in aqueous solution with phosphoric acid as catalyst to give photosensitive PVA-SbQ with different SbQ content and molecular weight.

In the working examples disclosed herein, GOx solutions were mixed into PVA-SbQ photo-sensitive polymer solutions. After exposure to UV light for an appropriate time period (from 10 seconds to 3 minutes, and typically from between 1 to 2 minutes), the PVA and SbQ were crosslinked so as to entrap GOx within the matrix. Sensors designed to include this material structure are observed to give a very stable Isig over time and further exhibit enhanced linearity of sensor response as compared to sensors formed with conventional materials. Without being bound by a specific theory or scientific principle, using this material structure within glucose sensors is believed to provide a better Isig quality in vivo because of the hydrogel being in close proximity to the electrode. Another advantage of the matrices disclosed herein (and methodology for making them) is that there is no need for crosslinkers and/or photo-initiators. Consequently, these methods and materials can avoid introducing potentially toxic substances to implantable sensor systems as well as provide better biocompatibility and an easier process control.

Typically, the amount of SbQ attached to PVA in glucose oxidase based sensors can vary from about 1 mol % to about 4.5 mol %. The relative photosensitivity of PVA-SbQ increased with increasing amount of bound SbQ in the case of high molecular weight (mw 77 kd to 79 kd), and decreased with decreasing molecular weight of PVA with about constant amount of bound SbQ (1.3 mol %). Photosensitive polymers were obtained when SbQ content reached about 2.63% in case of high MW (77 kd-79 kd) of PVA. The molecular weight of suitable polymers in such sensors is typically from 27 kd to 92 kd. Lower molecular weight macro porous polymer tends to be leachable from sensors (e.g. if not fully crosslinked). Higher molecular weight macro porous polymer are believed to be too viscous for use in glucose sensors (and may form a dense membrane structure that causes problematical glucose or oxygen mass-transport issues in glucose oxidase based sensors).

Polymer concentration and UV exposure condition and time also affect the cross linking density. Typically, the greater the SbQ content in the PVA-based polymer, the faster the UV cure and the greater the cross-linking density of the resultant polymer membrane. In addition a denser of membrane structure can be formed by increasing the MW of the polymer matrix. Suitable PVA-SbQ polymers useful in glucose sensor are typically designed to exhibit a neutral pH range. Embodiments of the invention can comprise analyte sensing layers (e.g. for use in an ambulatory glucose sensor) comprising a delineated amount of polymer, for example between 3% and 12.5% polyvinyl alcohol polymer.

Saponification is the hydrolysis of an ester under basic conditions to form an alcohol and the salt of a carboxylic acid (carboxylates). The degree of saponification is a factor that influences the solubility of a polyvinyl alcohol. In this context, another material parameter observed was the degree of saponification (DS %), that is the percentage of acetate groups present on the starting polymer (polyvinyl acetate) that are subsequently replaced by OH-groups. The higher the degree of the saponification degree, the higher the solubility and the speed of dissolution. In certain embodiments of the invention, PVA-SbQ polymers used in the sensor layers are selected to exhibit a degree of saponification of between 78% and 90%.

The SbQ content attached to the polymer determines the cross-linking density. Consequently, a higher crosslinking density could be helpful to retain GOx within the matrix and better structure integrity, but can also resulted in a less hydrophilic structure and other mass transport issues. Macroporous polymers tested that having a relatively high mw and relatively low SbQ content showed higher and more stable Isigs than control counterparts in a 5-day dog experiment. For example, a macroporous polymer having a SbQ content of 2.5% did show lower Isig level in vivo from the same sensor configuration having a SbQ content of 1.6%.

In the studies on working embodiments of the invention, it was discovered that certain constellations of elements unexpectedly produce sensors material properties that are very useful for amperometric glucose sensors that are disposed in in vivo environments. In this context, embodiments of the invention include a glucose sensor comprising a base layer and a working electrode, a reference electrode, and a counter electrode disposed on the base layer. This embodiment includes an analyte sensing layer that is between 1 μm and 5 μm in thickness disposed over the working electrode, wherein the analyte sensing layer comprises glucose oxidase entrapped within a PVA-SbQ polymer. In this optimized embodiment, the molecular weight of the polyvinyl alcohol in this layer is between 25 kilodaltons and 125 kilodaltons, and the SbQ in the layer comprises between 1 mol % and 4.5 mol % of the polymer. Typically the analyte sensing layer comprises PVA in an amount from 5% to 12% by weight; glucose oxidase in an amount from 10 KU/mL to 20 KU/mL; and human serum albumin in an amount from 1% to 5% by weight. As shown in FIGS. 4 and 5, glucose sensor embodiments having this constellation of elements exhibit Isig profiles having a greater long term stability as compared to control sensors that do not include this material. As shown in FIGS. 4 and 5, glucose sensor embodiments having this constellation of elements also exhibit Isig profiles having a greater sensitivity as compared to control sensors that do not include this material.

An exemplary working embodiment of the invention comprises a sensor designed to include a 4-pin distributed substrate on which the layers are disposed (e.g. 2 working electrodes, a counter electrode and a reference electrode). Embodiments of the invention include base designed to allow 360 degree sensing by fold, for example by using an analyte sensor comprising 20 ku/mL GOx layered onto a base substrate comprising planar sheet of a flexible material adapted to transition from a first configuration to a second configuration when the base substrate is folded to form a fixed bend (see, e.g. U.S. patent application Ser. No. 13/779,271). Such glucose sensors can be made by preparing a composition that combines a 20 ku/mL GOx 5% solution with a PVA-SbQ polymer (e.g. SAATI # MPP-Lab-2009) solution and then exposing this composition to UV light for 2 min at 7 mw/cm2 @360 nm wavelength, and 18 mw/cm2 @ 400 nm. One can then spray a protein layer comprising Human serum albumin on to the PVA-SbQ polymer composition layer (e.g. without using a chemical vapor deposition process). An adhesion promoting layer is then applied to the sensor stack using a dynamic spin coating process (“DSAP”) followed by a chemical vapor deposition process. The analyte modulating layer is then applied to the sensor stack (e.g. using a slot coating process).

### Illustrative Sensor Components and Systems of the Invention

In typical embodiments of the invention, electrochemical sensors are operatively coupled to a sensor input capable of receiving signals from the electrochemical sensor; and a processor coupled to the sensor input, wherein the processor is capable of characterizing one or more signals received from the electrochemical sensor. In certain embodiments of the invention, the electrical conduit of the electrode is coupled to a potentiostat. Optionally, a pulsed voltage is used to obtain a signal from an electrode. In certain embodiments of the invention, the processor is capable of comparing a first signal received from a working electrode in response to a first working potential with a second signal received from a working electrode in response to a second working potential. Optionally, the electrode is coupled to a processor adapted to convert data obtained from observing fluctuations in electrical current from a first format into a second format. Such embodiments include, for example, processors designed to convert a sensor current Input Signal (e.g. ISIG measured in nA) to a blood glucose concentration.

In many embodiments of the invention, the sensors comprise a biocompatible region adapted to be implanted in vivo. In some embodiments, the sensor comprises a discreet probe that pierces an in vivo environment. In embodiments of the invention, the biocompatible region can comprise a polymer that contacts an in vivo tissue. Optionally, the polymer is a hydrophilic polymer (e.g. one that absorbs water). In this way, sensors used in the systems of the invention can be used to sense a wide variety of analytes in different aqueous environments. In some embodiments of the invention, the electrode is coupled to a piercing member (e.g. a needle) adapted to be implanted in vivo. While sensor embodiments of the invention can comprise one or two piercing members, optionally such sensor apparatuses can include 3 or 4 or 5 or more piercing members that are coupled to and extend from a base element and are operatively coupled to 3 or 4 or 5 or more electrochemical sensors (e.g. microneedle arrays, embodiments of which are disclosed for example in U.S. Pat. Nos. 7,291,497 and 7,027,478, and U.S. patent Application No. 20080015494, the contents of which are incorporated by reference).

Embodiments of the invention include analyte sensor apparatus designed to utilize the compositions disclosed herein. Such apparatuses typically include a base on which an electrode is formed (e.g. an array of electrically conductive members configured to form a working electrode). Optionally this base comprises a plurality of indentations and the plurality of electrically conductive members are individually positioned within the plurality of indentations and the electrically conductive members comprise an electroactive surface adapted to sense fluctuations in electrical current at the electroactive surface.

In some embodiments of the invention where an electrode is formed from an array of electrically conductive members, the plurality of electrically conductive members are formed from shapes selected to avoid sharp edges and corners, electrode structures where electric charges can accumulate. In typical embodiments of the invention, the electrically conductive members can be formed to exhibit an ellipsoid geometry. For example, in some embodiments of the invention, the electrically conductive members comprise ellipses, circular discs, or combinations of ellipses and circular discs. Typically, such electrically conductive members are formed to have a diameter of at least 1 μm, for example, a diameter from 1 μm to 100 μm (e.g. circular discs having a diameter of 30, 40 or 50 μm). Optionally, the array comprises at least 5, 10, 20, 50 or 100 electrically conductive members.

In some embodiments of the invention, the array of electrically conductive members is coupled to a common electrical conduit (e.g. so that the conductive members of the array are not separately wired, and are instead electrically linked as a group). Optionally, the electrical conduit is coupled to a power source adapted to sense fluctuations in electrical current of the array of the working electrode. Typically the apparatus include a reference electrode; and a counter electrode. Optionally one or more of these electrodes also comprises a plurality of electrically conductive members disposed on the base in an array. In some embodiments, each of the electrically conductive members of the electrode (e.g. the counter electrode) comprises an electroactive surface adapted to sense fluctuations in electrical current at the electroactive surface; and the group of electrically conductive members are coupled to a power source (e.g. a potentiostat or the like).

In some embodiments of the invention, the apparatus comprises a plurality of working electrodes, counter electrodes and reference electrodes clustered together in units consisting essentially of one working electrode, one counter electrode and one reference electrode; and the clustered units are longitudinally distributed on the base layer in a repeating pattern of units. In some sensor embodiments, the distributed electrodes are organized/disposed within a flex-circuit assembly (i.e. a circuitry assembly that utilizes flexible rather than rigid materials). Such flex-circuit assembly embodiments provide an interconnected assembly of elements (e.g. electrodes, electrical conduits, contact pads and the like) configured to facilitate wearer comfort (for example by reducing pad stiffness and wearer discomfort).

Typically, the sensor electrodes of the invention are coated with a plurality of materials having properties that, for example, facilitate analyte sensing. In some embodiments of the invention, an analyte sensing layer is disposed over electrically conductive members, and includes an agent that is selected for its ability to detectably alter the electrical current at the working electrode in the presence of an analyte. In the working embodiments of the invention that are disclosed herein, the agent is glucose oxidase, a protein that undergoes a chemical reaction in the presence of glucose that results in an alteration in the electrical current at the working electrode. These working embodiments further include an analyte modulating layer disposed over the analyte sensing layer, wherein the analyte modulating layer modulates the diffusion of glucose as it migrates from an in vivo environment to the analyte sensing layer. In certain embodiments of the invention, the analyte modulating layer comprises a hydrophilic comb-copolymer having a central chain and a plurality of side chains coupled to the central chain, wherein at least one side chain comprises a silicone moiety. In certain embodiments of the invention, the analyte modulating layer comprises a blended mixture of: a linear polyurethane/polyurea polymer, and a branched acrylate polymer; and the linear polyurethane/polyurea polymer and the branched acrylate polymer are blended at a ratio of between 1:1 and 1:20 (e.g. 1:2) by weight %. Typically, this analyte modulating layer composition comprises a first polymer formed from a mixture comprising a diisocyanate; at least one hydrophilic diol or hydrophilic diamine; and a siloxane; that is blended with a second polymer formed from a mixture comprising: a 2-(dimethylamino) ethyl methacrylate; a methyl methacrylate; a polydimethyl siloxane monomethacryloxypropyl; a poly(ethylene oxide) methyl ether methacrylate; and a 2-hydroxyethyl methacrylate. Additional material layers can be included in such apparatuses. For example, in some embodiments of the invention, the apparatus comprises an adhesion promoting layer disposed between the analyte sensing layer and the analyte modulating layer.

Embodiments of the invention include dry plasma processes form making adhesion promoting (AP) layers in sensors comprising a plurality of layered materials (see, e.g. International Patent Application No. PCT/US2013/049138). The dry plasma processes disclosed PCT/US2013/049138 have a number of advantages over conventional wet chemistry processes used to form adhesion promoting layers, including reducing and/or eliminating the use of certain hazardous compounds, thereby reducing toxic wastes that can result from such processes. Embodiments of the invention also include adhesion promoting compositions formed from these processes, compositions that exhibit a combination of desirable material properties including relatively thin and highly uniform structural profiles.

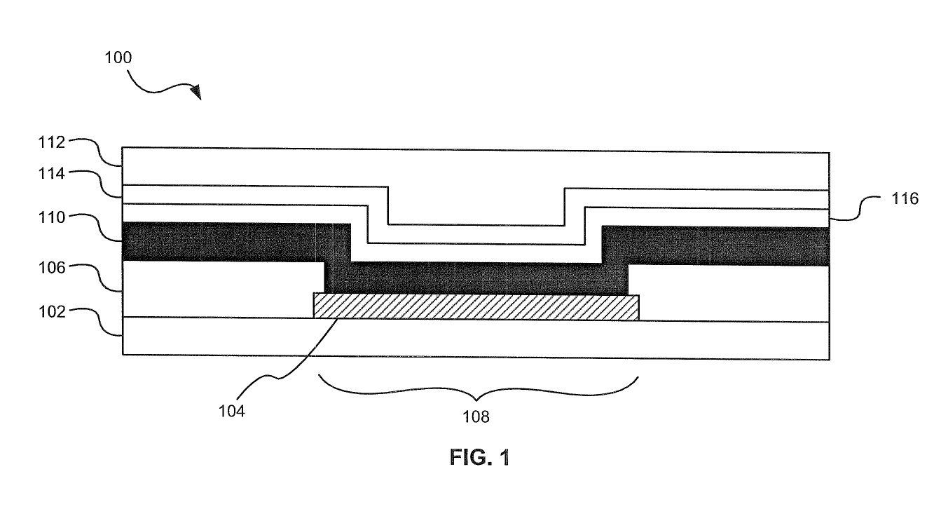

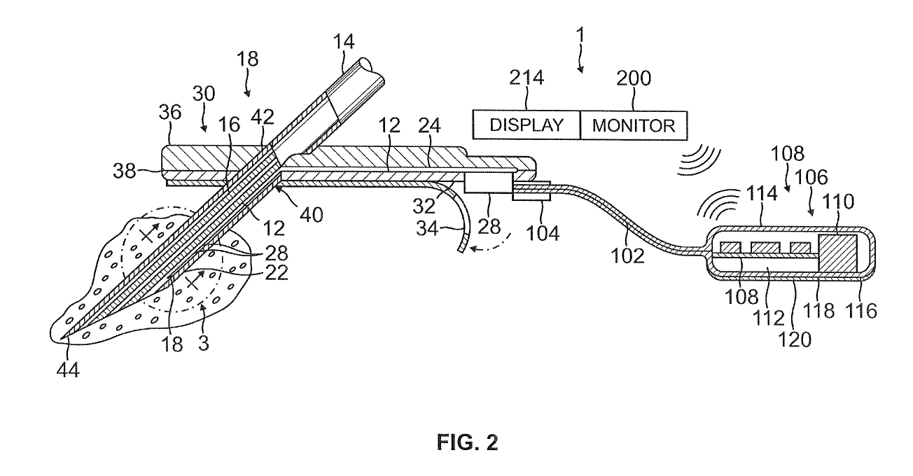

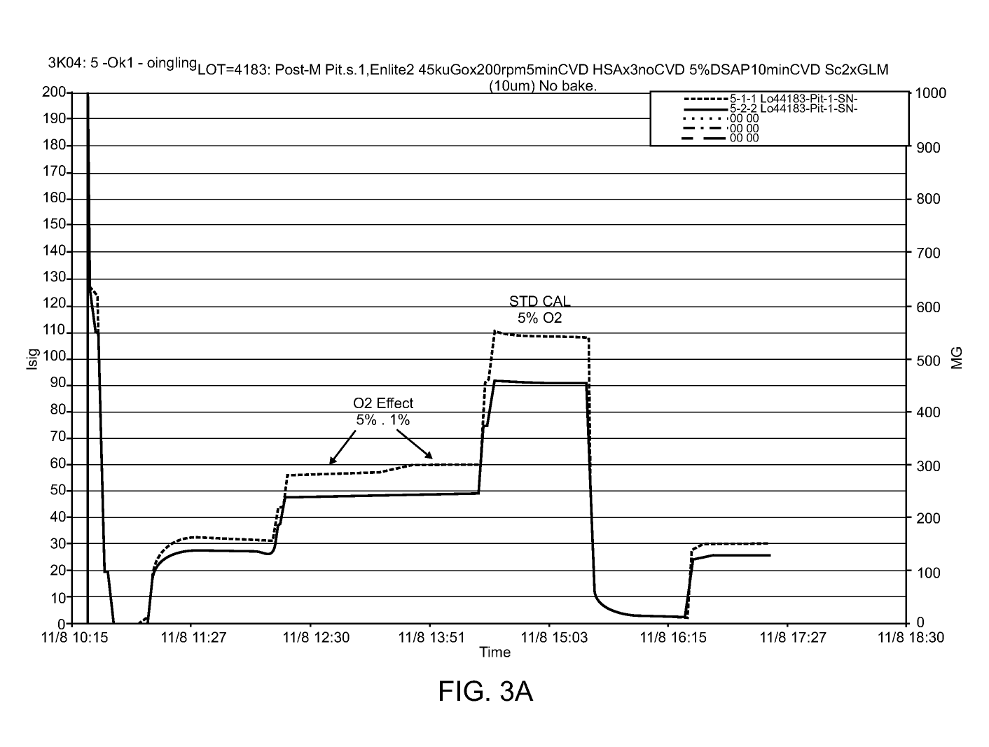

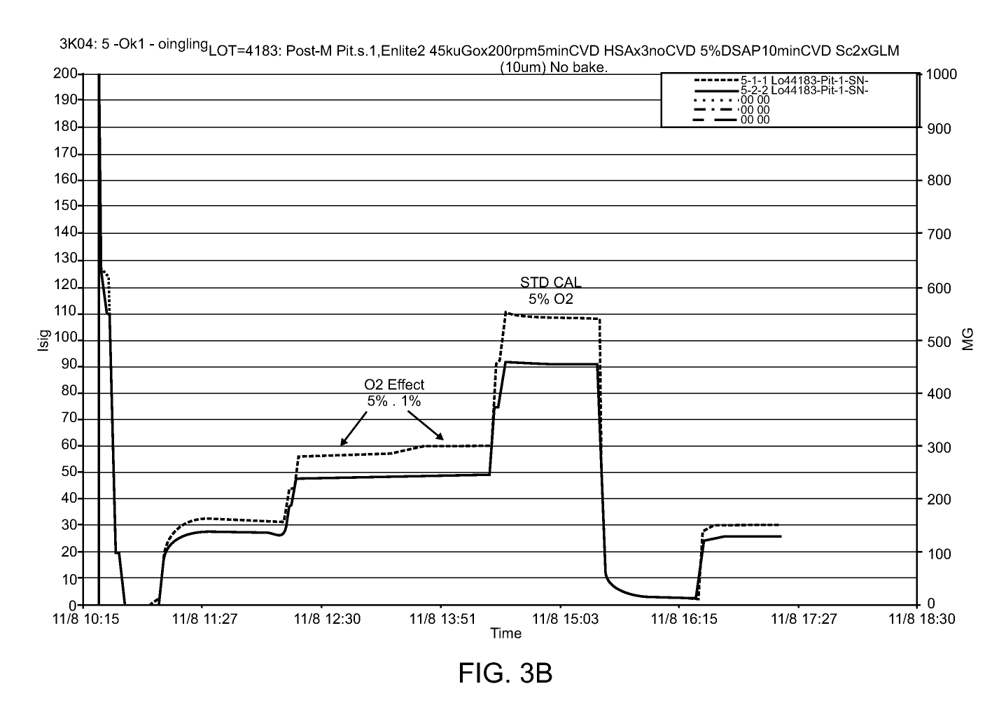

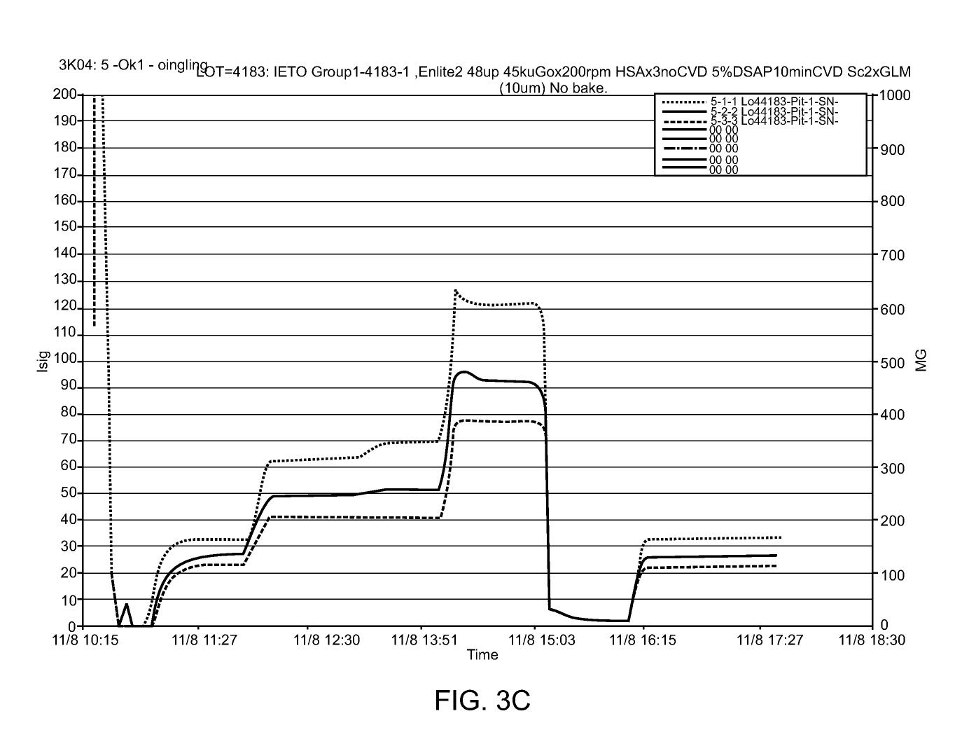

One sensor embodiment shown in FIG. 1 is a amperometric sensor 100 having a plurality of layered elements including a base layer 102, a conductive layer 104 (e.g. one comprising the plurality of electrically conductive members) which is disposed on and/or combined with the base layer 102. Typically the conductive layer 104 comprises one or more electrodes. An analyte sensing layer 110 (typically comprising an enzyme such as glucose oxidase) is disposed on one or more of the exposed electrodes of the conductive layer 104. A protein layer 116 disposed upon the analyte sensing layer 110. An analyte modulating layer 112 is disposed above the analyte sensing layer 110 to regulate analyte (e.g. glucose) access with the analyte sensing layer 110. An adhesion promoter layer 114 is disposed between layers such as the analyte modulating layer 112 and the analyte sensing layer 110 as shown in FIG. 2 in order to facilitate their contact and/or adhesion. This embodiment also comprises a cover layer 106 such as a polymer coating can be disposed on portions of the sensor 100. Apertures 108 can be formed in one or more layers of such sensors. Amperometric glucose sensors having this type of design are disclosed, for example, in U.S. Patent Application Publication Nos. 20070227907, 20100025238, 20110319734 and 20110152654, the contents of each of which are incorporated herein by reference.

Yet another embodiment of the invention is a method of sensing an analyte within the body of a mammal. Typically this method comprises implanting an analyte sensor having a PVA-SbQ compositions disclosed herein within the mammal (e.g. in the interstitial space of a diabetic individual), sensing an alteration in current at the working electrode in the presence of the analyte; and then correlating the alteration in current with the presence of the analyte, so that the analyte is sensed.

Embodiments of the invention also provide articles of manufacture and kits for observing a concentration of an analyte. In an illustrative embodiment, the kit includes a sensor comprising a composition as disclosed herein. In typical embodiments, the sensors are disposed in the kit within a sealed sterile dry package. Optionally the kit comprises an insertion device that facilitates insertion of the sensor. The kit and/or sensor set typically comprises a container, a label and an analyte sensor as described above. Suitable containers include, for example, an easy to open package made from a material such as a metal foil, bottles, vials, syringes, and test tubes. The containers may be formed from a variety of materials such as metals (e.g. foils) paper products, glass or plastic. The label on, or associated with, the container indicates that the sensor is used for assaying the analyte of choice. The kit and/or sensor set may include other materials desirable from a commercial and user standpoint, including buffers, diluents, filters, needles, syringes, and package inserts with instructions for use.

Specific aspects of embodiments of the invention are discussed in detail in the following sections.

### Typical Elements, Configurations and Analyte Sensor Embodiments of the Invention

### A. Typical Elements Found in of Embodiments of the Invention

FIG. 1 illustrates a cross-section of a typical sensor embodiment 100 of the present invention. This sensor embodiment is formed from a plurality of components that are typically in the form of layers of various conductive and non-conductive constituents disposed on each other according to art accepted methods and/or the specific methods of the invention disclosed herein. The components of the sensor are typically characterized herein as layers because, for example, it allows for a facile characterization of the sensor structure shown in FIG. 1. Artisans will understand however, that in certain embodiments of the invention, the sensor constituents are combined such that multiple constituents form one or more heterogeneous layers. In this context, those of skill in the art understand that the ordering of the layered constituents can be altered in various embodiments of the invention.

The embodiment shown in FIG. 1 includes a base layer 102 to support the sensor 100. The base layer 102 can be made of a material such as a metal and/or a ceramic and/or a polymeric substrate, which may be self-supporting or further supported by another material as is known in the art. Embodiments of the invention include a conductive layer 104 which is disposed on and/or combined with the base layer 102. Typically the conductive layer 104 comprises one or more electrically conductive elements that function as electrodes. An operating sensor 100 typically includes a plurality of electrodes such as a working electrode, a counter electrode and a reference electrode. Other embodiments may also include a plurality of working and/or counter and/or reference electrodes and/or one or more electrodes that performs multiple functions, for example one that functions as both as a reference and a counter electrode.

As discussed in detail below, the base layer 102 and/or conductive layer 104 can be generated using many known techniques and materials. In certain embodiments of the invention, the electrical circuit of the sensor is defined by etching the disposed conductive layer 104 into a desired pattern of conductive paths. A typical electrical circuit for the sensor 100 comprises two or more adjacent conductive paths with regions at a proximal end to form contact pads and regions at a distal end to form sensor electrodes. An electrically insulating cover layer 106 such as a polymer coating can be disposed on portions of the sensor 100. Acceptable polymer coatings for use as the insulating protective cover layer 106 can include, but are not limited to, non-toxic biocompatible polymers such as silicone compounds, polyimides, biocompatible solder masks, epoxy acrylate copolymers, or the like. In the sensors of the present invention, one or more exposed regions or apertures 108 can be made through the cover layer 106 to open the conductive layer 104 to the external environment and to, for example, allow an analyte such as glucose to permeate the layers of the sensor and be sensed by the sensing elements. Apertures 108 can be formed by a number of techniques, including laser ablation, tape masking, chemical milling or etching or photolithographic development or the like. In certain embodiments of the invention, during manufacture, a secondary photoresist can also be applied to the protective layer 106 to define the regions of the protective layer to be removed to form the aperture(s) 108. The exposed electrodes and/or contact pads can also undergo secondary processing (e.g. through the apertures 108), such as additional plating processing, to prepare the surfaces and/or strengthen the conductive regions.

In the sensor configuration shown in FIG. 1, an analyte sensing layer 110 is disposed on one or more of the exposed electrodes of the conductive layer 104. Typically, the analyte sensing layer 110 comprises an enzyme capable of producing and/or utilizing oxygen and/or hydrogen peroxide (for example glucose oxidase entrapped within a PVA-SbQ polymer). Optionally the enzyme in the analyte sensing layer is combined with a carrier protein such as human serum albumin, bovine serum albumin or the like. In an illustrative embodiment, an oxidoreductase enzyme such as glucose oxidase in the analyte sensing layer 110 reacts with glucose to produce hydrogen peroxide, a compound which then modulates a current at an electrode. As this modulation of current depends on the concentration of hydrogen peroxide, and the concentration of hydrogen peroxide correlates to the concentration of glucose, the concentration of glucose can be determined by monitoring this modulation in the current. In a specific embodiment of the invention, the hydrogen peroxide is oxidized at a working electrode which is an anode (also termed herein the anodic working electrode), with the resulting current being proportional to the hydrogen peroxide concentration. Such modulations in the current caused by changing hydrogen peroxide concentrations can by monitored by any one of a variety of sensor detector apparatuses such as a universal sensor amperometric biosensor detector or one of the other variety of similar devices known in the art such as glucose monitoring devices produced by Medtronic Diabetes.

In embodiments of the invention, the analyte sensing layer 110 can be applied over portions of the conductive layer or over the entire region of the conductive layer. Typically the analyte sensing layer 110 is disposed on the working electrode which can be the anode or the cathode. Optionally, the analyte sensing layer 110 is also disposed on a counter and/or reference electrode. Methods for generating a thin analyte sensing layer 110 include brushing the layer onto a substrate (e.g. the reactive surface of a platinum black electrode), as well as spin coating processes, dip and dry processes, low shear spraying processes, ink-jet printing processes, silk screen processes and the like. In certain embodiments of the invention, brushing is used to: (1) allow for a precise localization of the layer; and (2) push the layer deep into the architecture of the reactive surface of an electrode (e.g. platinum black produced by an electrodeposition process).

Typically, the analyte sensing layer 110 is coated and or disposed next to one or more additional layers. Optionally, the one or more additional layers includes a protein layer 116 disposed upon the analyte sensing layer 110. Typically, the protein layer 116 comprises a protein such as human serum albumin, bovine serum albumin or the like. Typically, the protein layer 116 comprises human serum albumin. In some embodiments of the invention, an additional layer includes an analyte modulating layer 112 that is disposed above the analyte sensing layer 110 to regulate analyte contact with the analyte sensing layer 110. For example, the analyte modulating membrane layer 112 can comprise a glucose limiting membrane, which regulates the amount of glucose that contacts an enzyme such as glucose oxidase that is present in the analyte sensing layer. Such glucose limiting membranes can be made from a wide variety of materials known to be suitable for such purposes, e.g., silicone compounds such as polydimethyl siloxanes, polyurethanes, polyurea cellulose acetates, Nafion, polyester sulfonic acids (e.g. Kodak AQ), hydrogels or any other suitable hydrophilic membranes known to those skilled in the art.

In typical embodiments of the invention, an adhesion promoter layer 114 is disposed between the analyte modulating layer 112 and the analyte sensing layer 110 as shown in FIG. 2 in order to facilitate their contact and/or adhesion. In a specific embodiment of the invention, an adhesion promoter layer 114 is disposed between the analyte modulating layer 112 and the protein layer 116 as shown in FIG. 2 in order to facilitate their contact and/or adhesion. The adhesion promoter layer 114 can be made from any one of a wide variety of materials known in the art to facilitate the bonding between such layers. Typically, the adhesion promoter layer 114 comprises a silane compound. In alternative embodiments, protein or like molecules in the analyte sensing layer 110 can be sufficiently crosslinked or otherwise prepared to allow the analyte modulating membrane layer 112 to be disposed in direct contact with the analyte sensing layer 110 in the absence of an adhesion promoter layer 114.

### B. Typical Analyte Sensor Constituents Used in Embodiments of the Invention

The following disclosure provides examples of typical elements/constituents used in sensor embodiments of the invention. While these elements can be described as discreet units (e.g. layers), those of skill in the art understand that sensors can be designed to contain elements having a combination of some or all of the material properties and/or functions of the elements/constituents discussed below (e.g. an element that serves both as a supporting base constituent and/or a conductive constituent and/or a matrix for the analyte sensing constituent and which further functions as an electrode in the sensor). Those in the art understand that these thin film analyte sensors can be adapted for use in a number of sensor systems such as those described below.

### Base Constituent

Sensors of the invention typically include a base constituent (see, e.g. element 102 in FIG. 1). The term “base constituent” is used herein according to art accepted terminology and refers to the constituent in the apparatus that typically provides a supporting matrix for the plurality of constituents that are stacked on top of one another and comprise the functioning sensor. In one form, the base constituent comprises a thin film sheet of insulative (e.g. electrically insulative and/or water impermeable) material. This base constituent can be made of a wide variety of materials having desirable qualities such as dielectric properties, water impermeability and hermeticity. Some materials include metallic, and/or ceramic and/or polymeric substrates or the like.

### Conductive Constituent

The electrochemical sensors of the invention typically include a conductive constituent disposed upon the base constituent that includes at least one electrode for contacting an analyte or its byproduct (e.g. oxygen and/or hydrogen peroxide) to be assayed (see, e.g. element 104 in FIG. 1). The term “conductive constituent” is used herein according to art accepted terminology and refers to electrically conductive sensor elements such as a plurality of electrically conductive members disposed on the base layer (e.g. so as to form a microarray electrode) and which are capable of measuring a detectable signal and conducting this to a detection apparatus. An illustrative example of this is a conductive constituent that forms a working electrode that can measure an increase or decrease in current in response to exposure to a stimuli such as the change in the concentration of an analyte or its byproduct as compared to a reference electrode that does not experience the change in the concentration of the analyte, a coreactant (e.g. oxygen) used when the analyte interacts with a composition (e.g. the enzyme glucose oxidase) present in analyte sensing constituent 110 or a reaction product of this interaction (e.g. hydrogen peroxide). Illustrative examples of such elements include electrodes which are capable of producing variable detectable signals in the presence of variable concentrations of molecules such as hydrogen peroxide or oxygen.

In addition to the working electrode, the analyte sensors of the invention typically include a reference electrode or a combined reference and counter electrode (also termed a quasi-reference electrode or a counter/reference electrode). If the sensor does not have a counter/reference electrode then it may include a separate counter electrode, which may be made from the same or different materials as the working electrode. Typical sensors of the present invention have one or more working electrodes and one or more counter, reference, and/or counter/reference electrodes. One embodiment of the sensor of the present invention has two, three or four or more working electrodes. These working electrodes in the sensor may be integrally connected or they may be kept separate. Optionally, the electrodes can be disposed on a single surface or side of the sensor structure. Alternatively, the electrodes can be disposed on a multiple surfaces or sides of the sensor structure (and can for example be connected by vias through the sensor material(s) to the surfaces on which the electrodes are disposed). In certain embodiments of the invention, the reactive surfaces of the electrodes are of different relative areas/sizes, for example a 1× reference electrode, a 2.6× working electrode and a 3.6× counter electrode.

### Interference Rejection Constituent

The electrochemical sensors of the invention optionally include an interference rejection constituent disposed between the surface of the electrode and the environment to be assayed. In particular, certain sensor embodiments rely on the oxidation and/or reduction of hydrogen peroxide generated by enzymatic reactions on the surface of a working electrode at a constant potential applied. Because amperometric detection based on direct oxidation of hydrogen peroxide requires a relatively high oxidation potential, sensors employing this detection scheme may suffer interference from oxidizable species that are present in biological fluids such as ascorbic acid, uric acid and acetaminophen. In this context, the term “interference rejection constituent” is used herein according to art accepted terminology and refers to a coating or membrane in the sensor that functions to inhibit spurious signals generated by such oxidizable species which interfere with the detection of the signal generated by the analyte to be sensed. Certain interference rejection constituents function via size exclusion (e.g. by excluding interfering species of a specific size). Examples of interference rejection constituents include one or more layers or coatings of compounds such as hydrophilic polyurethanes, cellulose acetate (including cellulose acetate incorporating agents such as poly(ethylene glycol), polyethersulfones, polytetra-fluoroethylenes, the perfluoronated ionomer Nafion™, polyphenylenediamine, epoxy and the like.

### Analyte Sensing Constituent

The electrochemical sensors of the invention include an analyte sensing constituent disposed on the electrodes of the sensor (see, e.g. element 110 in FIG. 1). In working embodiments of the invention disclosed herein, this constituent comprises glucose oxidase entrapped within a PVA-SbQ polymer. The term “analyte sensing constituent” is used herein according to art accepted terminology and refers to a constituent comprising a material that is capable of recognizing or reacting with an analyte whose presence is to be detected by the analyte sensor apparatus. Typically this material in the analyte sensing constituent produces a detectable signal after interacting with the analyte to be sensed, typically via the electrodes of the conductive constituent. In this regard the analyte sensing constituent and the electrodes of the conductive constituent work in combination to produce the electrical signal that is read by an apparatus associated with the analyte sensor. Typically, the analyte sensing constituent comprises an oxidoreductase enzyme capable of reacting with and/or producing a molecule whose change in concentration can be measured by measuring the change in the current at an electrode of the conductive constituent (e.g. oxygen and/or hydrogen peroxide), for example the enzyme glucose oxidase. An enzyme capable of producing a molecule such as hydrogen peroxide can be disposed on the electrodes according to a number of processes known in the art. The analyte sensing constituent can coat all or a portion of the various electrodes of the sensor. In this context, the analyte sensing constituent may coat the electrodes to an equivalent degree. Alternatively the analyte sensing constituent may coat different electrodes to different degrees, with for example the coated surface of the working electrode being larger than the coated surface of the counter and/or reference electrode.

Typical sensor embodiments of this element of the invention utilize an enzyme (e.g. glucose oxidase) that has been combined with a second protein (e.g. albumin) in a fixed ratio (e.g. one that is typically optimized for glucose oxidase stabilizing properties) and then applied on the surface of an electrode to form a thin enzyme constituent. In a typical embodiment, the analyte sensing constituent comprises a GOx and HSA mixture. In a typical embodiment of an analyte sensing constituent having GOx, the GOx reacts with glucose present in the sensing environment (e.g. the body of a mammal) and generates hydrogen peroxide.

As noted above, the enzyme and the second protein (e.g. an albumin) are typically treated to form a crosslinked matrix (e.g. by adding a cross-linking agent to the protein mixture). As is known in the art, crosslinking conditions may be manipulated to modulate factors such as the retained biological activity of the enzyme, its mechanical and/or operational stability. Illustrative crosslinking procedures are described in U.S. patent application Ser. No. 10/335,506 and PCT publication WO 03/035891 which are incorporated herein by reference. For example, an amine cross-linking reagent, such as, but not limited to, glutaraldehyde, can be added to the protein mixture. The addition of a cross-linking reagent to the protein mixture creates a protein paste. The concentration of the cross-linking reagent to be added may vary according to the concentration of the protein mixture. While glutaraldehyde is an illustrative crosslinking reagent, other cross-linking reagents may also be used or may be used in place of glutaraldehyde. Other suitable cross-linkers also may be used, as will be evident to those skilled in the art.

As noted above, in some embodiments of the invention, the analyte sensing constituent includes an agent (e.g. glucose oxidase) capable of producing a signal (e.g. a change in oxygen and/or hydrogen peroxide concentrations) that can be sensed by the electrically conductive elements (e.g. electrodes which sense changes in oxygen and/or hydrogen peroxide concentrations). However, other useful analyte sensing constituents can be formed from any composition that is capable of producing a detectable signal that can be sensed by the electrically conductive elements after interacting with a target analyte whose presence is to be detected. In some embodiments, the composition comprises an enzyme that modulates hydrogen peroxide concentrations upon reaction with an analyte to be sensed. Alternatively, the composition comprises an enzyme that modulates oxygen concentrations upon reaction with an analyte to be sensed. In this context, a wide variety of enzymes that either use or produce hydrogen peroxide and/or oxygen in a reaction with a physiological analyte are known in the art and these enzymes can be readily incorporated into the analyte sensing constituent composition. A variety of other enzymes known in the art can produce and/or utilize compounds whose modulation can be detected by electrically conductive elements such as the electrodes that are incorporated into the sensor designs described herein. Such enzymes include for example, enzymes specifically described in Table 1, pages 15-29 and/or Table 18, pages 111-112 of Protein Immobilization: Fundamentals and Applications (Bioprocess Technology, Vol 14) by Richard F. Taylor (Editor) Publisher: Marcel Dekker; Jan. 7, 1991) the entire contents of which are incorporated herein by reference.

### Protein Constituent

The electrochemical sensors of the invention optionally include a protein constituent disposed between the analyte sensing constituent and the analyte modulating constituent (see, e.g. element 116 in FIG. 1). The term “protein constituent” is used herein according to art accepted terminology and refers to constituent containing a carrier protein or the like that is selected for compatibility with the analyte sensing constituent and/or the analyte modulating constituent. In typical embodiments, the protein constituent comprises an albumin such as human serum albumin. The HSA concentration may vary between about 0.5%-30% (w/v). Typically the HSA concentration is about 1-10% w/v, and most typically is about 5% w/v. In alternative embodiments of the invention, collagen or BSA or other structural proteins used in these contexts can be used instead of or in addition to HSA. This constituent is typically crosslinked on the analyte sensing constituent according to art accepted protocols.

### Adhesion Promoting Constituent

The electrochemical sensors of the invention can include one or more adhesion promoting (AP) constituents (see, e.g. element 114 in FIG. 1). The term “adhesion promoting constituent” is used herein according to art accepted terminology and refers to a constituent that includes materials selected for their ability to promote adhesion between adjoining constituents in the sensor. Typically, the adhesion promoting constituent is disposed between the analyte sensing constituent and the analyte modulating constituent. Typically, the adhesion promoting constituent is disposed between the optional protein constituent and the analyte modulating constituent. The adhesion promoter constituent can be made from any one of a wide variety of materials known in the art to facilitate the bonding between such constituents and can be applied by any one of a wide variety of methods known in the art. Typically, the adhesion promoter constituent comprises a silane compound such as γ-aminopropyltrimethoxysilane.

### Analyte Modulating Constituent

The electrochemical sensors of the invention include an analyte modulating constituent disposed on the sensor (see, e.g. element 112 in FIG. 1). The term “analyte modulating constituent” is used herein according to art accepted terminology and refers to a constituent that typically forms a membrane on the sensor that operates to modulate the diffusion of one or more analytes, such as glucose, through the constituent. In certain embodiments of the invention, the analyte modulating constituent is an analyte-limiting membrane which operates to prevent or restrict the diffusion of one or more analytes, such as glucose, through the constituents. In other embodiments of the invention, the analyte-modulating constituent operates to facilitate the diffusion of one or more analytes, through the constituents. Optionally such analyte modulating constituents can be formed to prevent or restrict the diffusion of one type of molecule through the constituent (e.g. glucose), while at the same time allowing or even facilitating the diffusion of other types of molecules through the constituent (e.g. O₂).

With respect to glucose sensors, in known enzyme electrodes, glucose and oxygen from blood, as well as some interferants, such as ascorbic acid and uric acid, diffuse through a primary membrane of the sensor. As the glucose, oxygen and interferants reach the analyte sensing constituent, an enzyme, such as glucose oxidase, catalyzes the conversion of glucose to hydrogen peroxide and gluconolactone. The hydrogen peroxide may diffuse back through the analyte modulating constituent, or it may diffuse to an electrode where it can be reacted to form oxygen and a proton to produce a current that is proportional to the glucose concentration. The analyte modulating sensor membrane assembly serves several functions, including selectively allowing the passage of glucose therethrough (see, e.g. U.S. Patent Application No. 2011-0152654 the contents of which are incorporated by reference herein).

### Cover Constituent

The electrochemical sensors of the invention can include one or more cover constituents, which are typically electrically insulating protective constituents (see, e.g. element 106 in FIG. 1). Typically, such cover constituents can be in the form of a coating, sheath or tube and are disposed on at least a portion of the analyte modulating constituent. Acceptable polymer coatings for use as the insulating protective cover constituent can include, but are not limited to, non-toxic biocompatible polymers such as silicone compounds, polyimides, biocompatible solder masks, epoxy acrylate copolymers, or the like. Further, these coatings can be photo-imageable to facilitate photolithographic forming of apertures through to the conductive constituent. A typical cover constituent comprises spun on silicone. As is known in the art, this constituent can be a commercially available RTV (room temperature vulcanized) silicone composition. A typical chemistry in this context is polydimethyl siloxane (acetoxy based).

### C. Typical Analyte Sensor System Embodiments of the Invention

Embodiments of the sensor elements and sensors can be operatively coupled to a variety of other system elements typically used with analyte sensors (e.g. structural elements such as piercing members, insertion sets and the like as well as electronic components such as processors, monitors, medication infusion pumps and the like), for example to adapt them for use in various contexts (e.g. implantation within a mammal). One embodiment of the invention includes a method of monitoring a physiological characteristic of a user using an embodiment of the invention that includes an input element capable of receiving a signal from a sensor that is based on a sensed physiological characteristic value of the user, and a processor for analyzing the received signal. In typical embodiments of the invention, the processor determines a dynamic behavior of the physiological characteristic value and provides an observable indicator based upon the dynamic behavior of the physiological characteristic value so determined. In some embodiments, the physiological characteristic value is a measure of the concentration of blood glucose in the user. In other embodiments, the process of analyzing the received signal and determining a dynamic behavior includes repeatedly measuring the physiological characteristic value to obtain a series of physiological characteristic values in order to, for example, incorporate comparative redundancies into a sensor apparatus in a manner designed to provide confirmatory information on sensor function, analyte concentration measurements, the presence of interferences and the like.

Embodiments of the invention include devices which process display data from measurements of a sensed physiological characteristic (e.g. blood glucose concentrations) in a manner and format tailored to allow a user of the device to easily monitor and, if necessary, modulate the physiological status of that characteristic (e.g. modulation of blood glucose concentrations via insulin administration). An illustrative embodiment of the invention is a device comprising a sensor input capable of receiving a signal from a sensor, the signal being based on a sensed physiological characteristic value of a user; a memory for storing a plurality of measurements of the sensed physiological characteristic value of the user from the received signal from the sensor; and a display for presenting a text and/or graphical representation of the plurality of measurements of the sensed physiological characteristic value (e.g. text, a line graph or the like, a bar graph or the like, a grid pattern or the like or a combination thereof). Typically, the graphical representation displays real time measurements of the sensed physiological characteristic value. Such devices can be used in a variety of contexts, for example in combination with other medical apparatuses. In some embodiments of the invention, the device is used in combination with at least one other medical device (e.g. a glucose sensor).

An illustrative system embodiment consists of a glucose sensor, a transmitter and pump receiver and a glucose meter. In this system, radio signals from the transmitter can be sent to the pump receiver every 5 minutes to provide providing real-time sensor glucose (SG) values. Values/graphs are displayed on a monitor of the pump receiver so that a user can self monitor blood glucose and deliver insulin using their own insulin pump. Typically an embodiment of device disclosed herein communicates with a second medical device via a wired or wireless connection. Wireless communication can include for example the reception of emitted radiation signals as occurs with the transmission of signals via RF telemetry, infrared transmissions, optical transmission, sonic and ultrasonic transmissions and the like. Optionally, the device is an integral part of a medication infusion pump (e.g. an insulin pump). Typically in such devices, the physiological characteristic values include a plurality of measurements of blood glucose.

FIG. 2 provides a perspective view of one generalized embodiment of subcutaneous sensor insertion system and a block diagram of a sensor electronics device according to one illustrative embodiment of the invention. Additional elements typically used with such sensor system embodiments are disclosed for example in U.S. Patent Application No. 20070163894, the contents of which are incorporated by reference. FIG. 2 provides a perspective view of a telemetered characteristic monitor system 1, including a subcutaneous sensor set 10 provided for subcutaneous placement of an active portion of a flexible sensor 12, or the like, at a selected site in the body of a user. The subcutaneous or percutaneous portion of the sensor set 10 includes a hollow, slotted insertion needle 14 having a sharpened tip 44, and a cannula 16. Inside the cannula 16 is a sensing portion 18 of the sensor 12 to expose one or more sensor electrodes 20 to the user's bodily fluids through a window 22 formed in the cannula 16. The sensing portion 18 is joined to a connection portion 24 that terminates in conductive contact pads, or the like, which are also exposed through one of the insulative layers. The connection portion 24 and the contact pads are generally adapted for a direct wired electrical connection to a suitable monitor 200 coupled to a display 214 for monitoring a user's condition in response to signals derived from the sensor electrodes 20. The connection portion 24 may be conveniently connected electrically to the monitor 200 or a characteristic monitor transmitter 100 by a connector block 28 (or the like).

As shown in FIG. 2, in accordance with embodiments of the present invention, subcutaneous sensor set 10 may be configured or formed to work with either a wired or a wireless characteristic monitor system. The proximal part of the sensor 12 is mounted in a mounting base 30 adapted for placement onto the skin of a user. The mounting base 30 can be a pad having an underside surface coated with a suitable pressure sensitive adhesive layer 32, with a peel-off paper strip 34 normally provided to cover and protect the adhesive layer 32, until the sensor set 10 is ready for use. The mounting base 30 includes upper and lower layers 36 and 38, with the connection portion 24 of the flexible sensor 12 being sandwiched between the layers 36 and 38. The connection portion 24 has a forward section joined to the active sensing portion 18 of the sensor 12, which is folded angularly to extend downwardly through a bore 40 formed in the lower base layer 38. Optionally, the adhesive layer 32 (or another portion of the apparatus in contact with in vivo tissue) includes an anti-inflammatory agent to reduce an inflammatory response and/or anti-bacterial agent to reduce the chance of infection. The insertion needle 14 is adapted for slide-fit reception through a needle port 42 formed in the upper base layer 36 and through the lower bore 40 in the lower base layer 38. After insertion, the insertion needle 14 is withdrawn to leave the cannula 16 with the sensing portion 18 and the sensor electrodes 20 in place at the selected insertion site. In this embodiment, the telemetered characteristic monitor transmitter 100 is coupled to a sensor set 10 by a cable 102 through a connector 104 that is electrically coupled to the connector block 28 of the connector portion 24 of the sensor set 10.

In the embodiment shown in FIG. 2, the telemetered characteristic monitor 100 includes a housing 106 that supports a printed circuit board 108, batteries 110, antenna 112, and the cable 102 with the connector 104. In some embodiments, the housing 106 is formed from an upper case 114 and a lower case 116 that are sealed with an ultrasonic weld to form a waterproof (or resistant) seal to permit cleaning by immersion (or swabbing) with water, cleaners, alcohol or the like. In some embodiments, the upper and lower case 114 and 116 are formed from a medical grade plastic. However, in alternative embodiments, the upper case 114 and lower case 116 may be connected together by other methods, such as snap fits, sealing rings, RTV (silicone sealant) and bonded together, or the like, or formed from other materials, such as metal, composites, ceramics, or the like. In other embodiments, the separate case can be eliminated and the assembly is simply potted in epoxy or other moldable materials that is compatible with the electronics and reasonably moisture resistant. As shown, the lower case 116 may have an underside surface coated with a suitable pressure sensitive adhesive layer 118, with a peel-off paper strip 120 normally provided to cover and protect the adhesive layer 118, until the sensor set telemetered characteristic monitor transmitter 100 is ready for use.

In the illustrative embodiment shown in FIG. 2, the subcutaneous sensor set 10 facilitates accurate placement of a flexible thin film electrochemical sensor 12 of the type used for monitoring specific blood parameters representative of a user's condition. The sensor 12 monitors glucose levels in the body, and may be used in conjunction with automated or semi-automated medication infusion pumps of the external or implantable type as described in U.S. Pat. Nos. 4,562,751; 4,678,408; 4,685,903 or 4,573,994, to control delivery of insulin to a diabetic patient.

In the illustrative embodiment shown in FIG. 2, the sensor electrodes 10 may be used in a variety of sensing applications and may be configured in a variety of ways. For example, the sensor electrodes 10 may be used in physiological parameter sensing applications in which some type of biomolecule is used as a catalytic agent. For example, the sensor electrodes 10 may be used in a glucose and oxygen sensor having a glucose oxidase enzyme catalyzing a reaction with the sensor electrodes 20. The sensor electrodes 10, along with a biomolecule or some other catalytic agent, may be placed in a human body in a vascular or non-vascular environment. For example, the sensor electrodes 20 and biomolecule may be placed in a vein and be subjected to a blood stream, or may be placed in a subcutaneous or peritoneal region of the human body.

In the embodiment of the invention shown in FIG. 2, the monitor of sensor signals 200 may also be referred to as a sensor electronics device 200. The monitor 200 may include a power source, a sensor interface, processing electronics (i.e. a processor), and data formatting electronics. The monitor 200 may be coupled to the sensor set 10 by a cable 102 through a connector that is electrically coupled to the connector block 28 of the connection portion 24. In an alternative embodiment, the cable may be omitted. In this embodiment of the invention, the monitor 200 may include an appropriate connector for direct connection to the connection portion 104 of the sensor set 10. The sensor set 10 may be modified to have the connector portion 104 positioned at a different location, e.g., on top of the sensor set to facilitate placement of the monitor 200 over the sensor set.

While the analyte sensor and sensor systems disclosed herein are typically designed to be implantable within the body of a mammal, the inventions disclosed herein are not limited to any particular environment and can instead be used in a wide variety of contexts, for example for the analysis of most in vivo and in vitro liquid samples including biological fluids such as interstitial fluids, whole-blood, lymph, plasma, serum, saliva, urine, stool, perspiration, mucus, tears, cerebrospinal fluid, nasal secretion, cervical or vaginal secretion, semen, pleural fluid, amniotic fluid, peritoneal fluid, middle ear fluid, joint fluid, gastric aspirate or the like. In addition, solid or desiccated samples may be dissolved in an appropriate solvent to provide a liquid mixture suitable for analysis.

TABLE 1
Thermal resistance (polymer films after aging at 60° C. and 100% Relative humidity oven)

| | | Day 0 | Mw after aging in humid at 60° C. (kD) | | | | | |
|---|---|---|---|---|---|---|---|---|
| GLM | Lot # | (kD) | Day 1 | Day 2 | Day 3 | Day 6 | Day 7 | % Loss |
| Control | 16601-24 | 140 | 110 | 96 | 85 | 63 | 62 | 56% |
| Control | 17183-65 | 146 | 103 | 86 | 70 | 57 | 47 | 68% |
| Control + 10% MDI | 17580-17a | 144 | 126 | 114 | 104 | 95 | 87 | 40% |
| Control + 10% MDI | 17580-17b | 140 | 131 | 125 | 114 | 100 | 96 | 31% |

It is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims. In the description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

1. A method of making an analyte sensor apparatus comprising:
disposing a working electrode, a reference electrode, and a counter electrode on a base layer;
disposing an analyte sensing layer over the working electrode, wherein:
the analyte sensing layer comprises glucose oxidase disposed within a polyvinyl alcohol polymer comprising N-methyl-4(4′-formylstyryl)pyridinium (PVA-SbQ);
the PVA-SbQ polymer is selected for an ability to inhibit damage to glucose oxidase by ethylene oxide; and
disposing an analyte modulating layer disposed over the analyte sensing layer, wherein the analyte modulating layer modulates the diffusion of analyte therethrough;
so that an analyte sensor apparatus is made.

2. The method of claim 1, wherein the analyte sensing layer:
(a) comprises PVA-SbQ in an amount from 5% to 15% by weight;
(b) comprises glucose oxidase in an amount from 10 KU/mL to 20 KU/mL;
(c) comprises polyvinyl alcohol having a molecular weight from 25 kilodaltons to 125 kilodaltons;
(d) comprises 1.0% to 4.0% N-methyl-4(4′-formylstyryl)pyridinium; and/or
(e) is between 4 and 12 microns in thickness.

3. The method of claim 1, wherein the analyte sensor apparatus comprises a further layer disposed over the analyte sensing layer, wherein the further layer:
(a) comprises PVA-SbQ;
(b) comprises a hydrophilic polyurethane;
(c) does not include an albumin; and/or
(d) is between 1 and 3 microns in thickness.

4. The method of claim 1, wherein the analyte modulating layer comprises:
(1) a polyurethane/polyurea polymer formed from a mixture comprising:
(a) a diisocyanate;
(b) a hydrophilic polymer comprising a hydrophilic diol or hydrophilic diamine; and
(c) a siloxane having an amino, hydroxyl or carboxylic acid functional group at a terminus; and/or
(2) a branched acrylate polymer formed from a mixture comprising:
(a) a butyl, propyl, ethyl or methyl-acrylate;
(b) an amino-acrylate;
(c) a siloxane-acrylate; and
(d) a poly(ethylene oxide)-acrylate.

5. The method of claim 1, wherein the analyte modulating layer is formed from a composition comprising a diisocyanate having a phenyl moiety.

6. A method of inhibiting damage to glucose oxidase caused by ethylene oxide vapor during a sterilization process, the method comprising disposing the glucose oxidase within a matrix comprising a polyvinyl alcohol polymer comprising N-methyl-4(4′-formylstyryl)pyridinium (PVA-SbQ), so that damage to glucose oxidase is inhibited.

7. The method of claim 6, wherein the glucose oxidase is disposed within an analyte sensor apparatus, wherein the analyte sensor apparatus comprises:
a base layer;
a working electrode, a reference electrode, and a counter electrode disposed on the base layer;
an analyte sensing layer disposed over the working electrode, wherein the analyte sensing layer comprises the glucose oxidase disposed within the PVA-SbQ; and
an analyte modulating layer disposed over the analyte sensing layer, wherein the analyte modulating layer modulates the diffusion of analyte therethrough.

8. The method of claim 7, wherein the analyte sensing layer:
(a) comprises PVA-SbQ in an amount from 5% to 15% by weight;
(b) comprises glucose oxidase in an amount from 10 KU/mL to 20 KU/mL;
(c) comprises polyvinyl alcohol having a molecular weight from 25 kilodaltons to 125 kilodaltons;
(d) comprises 1.0% to 4.0% N-methyl-4(4′-formylstyryl)pyridinium; and/or
(e) is between 4 and 12 microns in thickness.

9. The method of claim 6, wherein the analyte sensor apparatus comprises a further layer disposed over the analyte sensing layer, wherein the further layer:
(a) comprises PVA-SbQ;
(b) comprises a hydrophilic polyurethane;
(c) does not include an albumin; and/or
(d) is between 1 and 3 microns in thickness.

10. A method of inhibiting microbial growth on an analyte sensor apparatus, the method comprising:
exposing the analyte sensor apparatus to an ethylene oxide vapor so as to contact a microorganism present on the analyte sensor apparatus or a container in which the analyte sensor apparatus is disposed; and
allowing the ethylene oxide to alkylate DNA of the microorganism, thereby inhibiting microbial growth,
wherein the analyte sensor apparatus comprises:
a base layer;
a working electrode, a reference electrode, and a counter electrode disposed on the base layer;
an analyte sensing layer disposed over the working electrode, wherein the analyte sensing layer comprises glucose oxidase disposed within a polyvinyl alcohol polymer comprising N-methyl-4(4′-formylstyryl)pyridinium (PVA-SbQ); and
an analyte modulating layer disposed over the analyte sensing layer, wherein the analyte modulating layer modulates the diffusion of analyte therethrough.

11. The method of claim 10, wherein the analyte sensing layer:
(a) comprises PVA-SbQ in an amount from 5% to 15% by weight;
(b) comprises glucose oxidase in an amount from 10 KU/mL to 20 KU/mL;
(c) comprises polyvinyl alcohol having a molecular weight from 25 kilodaltons to 125 kilodaltons;
(d) comprises 1.0% to 4.0% N-methyl-4(4′-formylstyryl)pyridinium; and/or
(e) is between 4 and 12 microns in thickness.

12. The method of claim 10, wherein the analyte sensor apparatus comprises a further layer disposed over the analyte sensing layer, wherein the further layer:
(a) comprises PVA-SbQ;
(b) comprises a hydrophilic polyurethane;
(c) does not include an albumin; and/or
(d) is between 1 and 3 microns in thickness.

13. The method of claim 10, wherein:
the method uses ethylene oxide vapor in a concentration range from 50 to 1,500 mg/L;
the method uses humidity in a range from 30% to 90%;
the methods is performed at a temperature from 25-55° C.; or
the method is performed for at least 2 hours.

14. The method of claim 10, wherein the analyte modulating layer is formed from a diisocyanate comprising a phenyl moiety.

15. A composition of matter comprising:
(1) analyte sensor apparatus having:
a base layer;
a working electrode, a reference electrode, and a counter electrode disposed on the base layer;
an analyte sensing layer disposed over the working electrode, wherein the analyte sensing layer comprises glucose oxidase entrapped within:
(a) a polyvinyl alcohol polymer comprising N-methyl-4(4′-formylstyryl)pyridinium (SbQ); or (b) a hydrophilic polyurethane;
an analyte modulating layer disposed over the analyte sensing layer, wherein the analyte modulating layer modulates the diffusion of analyte therethrough; and
(2)ethylene oxide vapor.

16. The composition of claim 15, wherein the analyte sensing layer:
(a) comprises PVA-SbQ in an amount from 5% to 15% by weight;
(b) comprises glucose oxidase in an amount from 10 KU/mL to 20 KU/mL;
(c) comprises polyvinyl alcohol having a molecular weight from 25 kilodaltons to 125 kilodaltons;
(d) comprises 1.0% to 4.0% N-methyl-4(4′-formylstyryl)pyridinium; and/or
(e) is between 4 and 12 microns in thickness.

17. The composition of claim 15, wherein the composition does not comprise an albumin.

18. The composition of claim 15, wherein the analyte modulating layer comprises:
polyurethane/polyurea polymer formed from a mixture comprising:
(a) a diisocyanate;
(b) a hydrophilic polymer comprising a hydrophilic diol or hydrophilic diamine; and
(c) a siloxane having an amino, hydroxyl or carboxylic acid functional group at a terminus; and/or
a branched acrylate polymer formed from a mixture comprising:
(a) a butyl, propyl, ethyl or methyl-acrylate;
(b) an amino-acrylate;
(c) a siloxane-acrylate; and

19. The composition of claim 15, wherein the analyte sensor apparatus comprises a further layer disposed over the analyte sensing layer, wherein the further layer:
(a) comprises PVA-SbQ;
(b) comprises a hydrophilic polyurethane;
(c) does not include an albumin; and/or
(d) is between 1 and 3 microns in thickness.

20. The composition of claim 15, wherein the analyte sensing layer comprises glucose oxidase trapped within a hydrophilic polyurethane.
